(19) United States
(12) Patent Application Publication
(10) Pub. No.: US 2007/0116600 A1
(43) Pub. Date: May 24, 2007

(54) Detection device and methods associated therewith

(76) Inventors: Manish Swarnaraj Kochar, Rockville, MD (US); Charles Quentin Davis, Frederick, MD (US); Jonathan K. Leland, Silver Spring, MD (US); Richard Manteuffel, Laytonsville, MD (US); Richard A. Cook, Deerwood, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/474,099

(22) Filed: Jun. 23, 2006

Related U.S. Application Data
(60) Provisional application No. 60/693,049, filed on Jun. 23, 2005.

(51) Int. Cl. G01N 33/00

(52) U.S. Cl. 422/65

(57) ABSTRACT

Detection devices and methods associated therewith are provided. In some embodiments, the detection system can be adapted to measure one or more analytes of interest possibly present in a sample through the use of binding reactions.

### CROSS-REFERENCE

This application claims the benefit of U.S. Provisional Application No. 60/693,049, filed Jun. 23, 2005, which is herein incorporated by reference in its entirety.

### TECHNICAL FIELD

The present invention relates to a detection device and methods associated with the detection device.

### BACKGROUND

In the medical, environmental, biodefense, and food safety communities, immunodiagnostic testing can provide a simple assessment and rapid identification of diseases and contaminants that are harmful to individuals and society. To monitor and prevent the occurrence of protracted illness and/or endemic disease, there is a need for simple screening and confirmatory assays that provide qualitative, semi-quantitative, and quantitative assessment for the detection of analytes, such as an antigen in a clinical specimen, soil or water sample, or food. In addition, due to the realization of the threat of national terrorism in recent years, many diagnostic tests are designed to be performed at satellite sites rather than established laboratories. Further, the availability of rapid and reliable on-site immunodiagnostic tests can improve the quality and timeliness of appropriate medical treatment.

There remains a need to develop new methods and apparatus for reliable and easy to use diagnostic assays.

### SUMMARY OF THE INVENTION

Consistent with embodiments of the present invention, detection devices and methods associated therewith are provided.

In one embodiment, a biological detection system used to measure the presence and/or quantity for one or more analytes of interest in a sample through the use of binding reactions can be provided. The detection system can comprise a detector configured to detect a label used in the binding reactions. The detection system can further comprise a holder configured to hold at least one multi-well reagent container. The reagent container can include binding reagents for a plurality of binding reactions. The detection system can further comprise a holder for a sample container and a probe. The probe can be configured to at least distribute a known amount of sample into at least one of the at least one multi-well reagent containers. The system can further comprise one pump fluidically connected to the probe. The detection system can also comprise a liquid-level detector for determining the presence of liquid and/or the liquid level in the sample container and/or the multi-well reagent container. In another embodiment, the detection system may measure the presence and/or quantity of a single analyte of interest in multiple samples. Thus, samples obtained from one or more sources may undergo anaylsis in a single detection system. Also disclosed herein are biological detection systems having two or more magnetic capture zones. The two or more magnetic capture zones may be fluidically connected to collect and release magnetizable beads.

In another embodiment, multi-well reagent containers are disclosed. A multi-well reagent container according to the principles disclosed herein may include one or more vessels and a receptable. The one or more vessels may be held in the receptacle with an attachment retention member and the one or more vessels may be physically separate parts.

In another embodiment, the invention can comprise a method to measure an analyte of interest possibly present in liquid in a sample container. The method can comprise forming a composition in a well. The composition can comprise a sample of optionally processed liquid from the sample container and binding reagents comprising a plurality of magnetizable beads, a plurality of labels, and a plurality of reagents specific for the one or more analytes of interest. The method can further comprise incubating the composition to form complexes among the label, analyte of interest, and the magnetizable bead. The method can also comprise separating the non-complexed label and sample matrix from the complexed label using a method comprising (i) aspirating the incubated composition from the well; (ii) capturing the magnetizable beads with a magnet; and (iii) dispensing the composition that is not magnetically captured into a waste location. The method can further comprise releasing the captured magnetizable beads and transporting the magnetizable beads to a measurement zone. The method can also comprise detecting the label to measure the concentration of the analyte of interest.

It is to be understood that both the foregoing general description and the following detailed description are exemplary and explanatory only and are not restrictive of the invention as claimed. The foregoing background and summary are not intended to provide any independent limitations on the claimed invention.

### BRIEF DESCRIPTION OF THE DRAWINGS

FIG. 1 is a schematic representation of one exemplary embodiment of a detection system consistent with the principles of the present invention.

FIG. 2 is an isometric view of an exemplary multi-well reagent container holder consistent with the principles of the present invention.

FIG. 3 is an isometric view of an exemplary multi-well reagent container consistent with the principles of the present invention.

FIG. 4A is a partial isometric view of a probe, tubing and a magnet set consistent with the principles of the present invention, showing the magnet set in a position proximal to the tubing.

FIG. 4B is a partial isometric view of a probe, tubing and a magnet set consistent with the principles of the present invention, showing the magnet set in a position distal to the tubing.

FIG. 5A is a partial top view of tubing and a magnet set consistent with the principles of the present invention, showing the magnet set in a position proximal to the tubing.

FIG. 5B is a partial top view of tubing and a magnet set consistent with the principles of the present invention, showing the magnet set in a position distal to the tubing.

FIG. 6 is a block diagram depicting a technique consistent with the principles of the present invention for detecting the liquid level in a sample container utilizing a probe and an oscillator.

FIG. 7 illustrates various embodiments of a filter well consistent with the principles of the present invention.

FIGS. 8A and 8B are graphs illustrating exemplary velocity and acceleration profiles, respectively, for controlling a tray consistent with the principles of the present invention.

FIG. 9 illustrates a filter inside a probe connected to a pump consistent with the principles of the present invention.

FIG. 10 is an isometric view of another exemplary multi-well reagent container consistent with the principles of the present invention.

FIG. 11 is a top view of an exemplary multi-well reagent container consistent with the principles of the present invention.

FIG. 12 is an isometric view of an exemplary sample container holder consistent with the principles of the present invention.

FIGS. 13A, 13B and 13C are isometric views of exemplary sample container holders consistent with the principles of the present invention.

FIG. 14 is a graph of the results of replicate measurements of unfiltered TSH assays performed with prewashing and without prewashing. FIG. 14 illustrates 96 assays from a single multi-well container having 96 wells. Half of the assays were prewashed, and half were not.

FIG. 15 is a schematic representation of a detection system.

FIGS. 16A and 16B show two embodiments for a probe, tubing, and magnet set.

FIGS. 17A, 17B, 17C, and 17D show an embodiment for a multi-well reagent container holder and embodiments for snap-fit reagent containers.

FIGS. 18A, 18B, and 18C show embodiments for holders for sample containers.

FIGS. 19A and 19B show two views of an embodiment for a multiwell reagent container including a filter cartridge.

### DETAILED DESCRIPTION

The following description refers to the accompanying drawings in which, in the absence of a contrary representation, the same numbers in different drawings represent similar elements. The implementations in the following description do not represent all implementations consistent with principles of the claimed invention. Instead, they are merely some examples of systems and methods consistent with those principles. It is to be understood that both the foregoing general description and the following detailed description are exemplary and explanatory only and are not restrictive of the invention as claimed.

I. Definitions

In order to more clearly understand the invention, certain terms are defined as follows:

The term “aliphatic”, as used herein, is defined as in The American Heritage® Dictionary of the English Language, Fourth Edition Copyright © 2000 and encompasses organic chemical compounds in which the carbon atoms are linked in open chains. The open chains range from 1 to 20 carbon atoms, from 1 to 13 carbon atoms, or from 1 to 6 carbon atoms. The points of unsaturation for an aliphatic group may range from 1 to 10, from 1 to 6, or from 1 to 3. The number of carbon atoms in an aliphatic group can be indicated by a subscript on a “C”; for example, “C₃ aliphatic” represents an aliphatic group comprising 3 carbon atoms. Likewise, ranges can be expressed in the subscript. For example “C₁₋₁₀ aliphatic” encompasses aliphatic groups of from 1 to 10 carbon atoms inclusive. Examples of aliphatic groups include, but are not limited to, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, ethene, propene, ethyne, butene, propyne, and butyne. When an aliphatic group having a specific number of carbons is named using the subscripted C notation, all isomers having that number of carbons are intended to be encompassed. Aliphatic groups may be optionally substituted by at least one hydrophilic functional group, as defined herein. In addition, aliphatic groups useful as ECL moieties and as ECL coreactants may also comprise additional functional groups and may have a single (i.e., monodentate ligand) or multiple (i.e., bidentate or polydentate ligands) points of attachment. Such aliphatic groups are well known in the art and are described in Electrogenerated Chemiluminescence, Bard, Editor, Marcel Dekker, (2004); Knight, A and Greenway, G. Analyst 119:879-890 1994.

The term “hydrophilic functional group” refers to a functional group that facilitates or that increases the solubility of a molecule in water. Examples include, but are not limited to, groups such as hydroxyl (—OH), aldehyde (—C(O)H), hydroxycarbonyl (—C(OH)(C═O)H), amino (—NH₂), aminocarbonyl (—CONH₂), amidine (—C(═NH)NH₂), imino (—C═NH), cyano (—CN), nitro (—NO₂), nitrate (—NO₃), sulfate (—SO₄), sulfonate (—SO₃H), phosphate (—PO₄), phosphonate (—PH₂O₃), silicate (—SiHO₃), carboxylate (—COOH), borate (—BH₂O₃), guanidinium (—HN—C(═NH)—NH₂), carbamide (—HNC(O)NH₂), carbamate (—HNC(O)NH₂), carbonate (—CO₃), sulfamide (—S(O)₂NH₂), silyl (—SiH₃ and/or —Si(OH)₃), siloxy (—OSiH₃ and/or —OSi(OH)₃), and amide.

The term “dry composition” or “dry” as used herein, means that the composition has a moisture content of less than or equal to about 5% by weight, relative to the total weight of the composition. Examples of dry compositions include compositions that have a moisture content of less than or equal to about 3% by weight, relative to the total weight of the composition and compositions that have a moisture content ranging from about 1% to about 3% by weight, relative to the total weight of the composition.

The term “binding partner,” as used herein, means a substance that can bind specifically to an analyte of interest. In general, specific binding is characterized by a relatively high affinity and a relatively low to moderate capacity. Nonspecific binding usually has a low affinity with a moderate to high capacity. Typically, binding is considered specific when the affinity constant Kₐ is higher than about 10⁶M⁻¹. For example, binding may be considered specific when the affinity constant Kₐ is higher than about 10⁸M⁻¹. A higher affinity constant indicates greater affinity, and thus typically greater specificity. For example, antibodies typically bind antigens with an affinity constant in the range of 10⁶M⁻¹ to 10⁹M⁻¹ or higher.

Examples of binding partners include complementary nucleic acid sequences (e.g., two DNA sequences which hybridize to each other; two RNA sequences which hybridize to each other; a DNA and an RNA sequence which hybridize to each other), an antibody and an antigen, a receptor and a ligand (e.g., TNF and TNFr-1, CD142 and Factor Vlla, B7-2 and CD28, HIV-1 and CD4, ATR/TEM8 or CMG and the protective antigen moiety of anthrax toxin), an enzyme and a substrate, or a molecule and a binding protein (e.g., vitamin B12 and intrinsic factor, folate and folate binding protein).

Examples of binding partners include antibodies. The term “antibody,” as used herein, means an immunoglobulin or a part thereof, and encompasses any polypeptide (with or without further modification by sugar moieties (mono and polysaccharides)) comprising an antigen-binding site regardless of the source, method of production, or other characteristics. The term includes, for example, polyclonal, monoclonal, monospecific, polyspecific, humanized, single-chain, chimeric, synthetic, recombinant, hybrid, mutated, and CDR-grafted antibodies as well as fusion proteins. A part of an antibody can include any fragment which can bind antigen, including but not limited to Fab, Fab′, F(ab′)₂, Facb, Fv, ScFv, Fd, V_{H}, and V_{L}.

A large number of monoclonal antibodies that bind to various analytes of interest are available, as exemplified by the listings in various catalogs, such as: Biochemicals and Reagents for Life Science Research, Sigma-Aldrich Co., P.O. Box 14508, St. Louis, Mo., 63178 (1999); the Life Technologies Catalog, Life Technologies, Gaithersburg, Md.; and the Pierce Catalog, Pierce Chemical Company, P.O. Box 117, Rockford, Ill. 61105 (1994).

Other exemplary, possibly monoclonal, antibodies include those that bind specifically to β-actin, DNA, digoxin, insulin, progesterone, human leukocyte markers, human interleukin-10, human interferon, human fibrinogen, p53, hepatitis B virus or a portion thereof, HIV virus or a portion thereof, tumor necrosis factor, or FK-506. In certain embodiments, the monoclonal antibody is chosen from antibodies that bind specifically to at least one of T4, T3, free T3, free T4, TSH (thyroid-stimulating hormone), thyroglobulin, TSH receptor, prolactin, LH (luteinizing hormone), FSH (follicle stimulating hormone), testosterone, progesterone, estradiol, hCG (human Chorionic Gondaotropin), hCG+β, SHBG (sex hormone-binding globulin), DHEA-S (dehydroepiandrosterone sulfate), hGH (human growth hormone), ACTH (adrenocorticotropic hormone), cortisol, insulin, ferritin, folate, RBC (red blood cell) folate, vitamin B12, vitamin D, C-peptide, troponin T, CK-MB (creatine kinase-myoglobin), myoglobin, pro-BNP (brain natriuretic peptide), HbsAg (hepatitis B surface antigen), HbeAg (hepatitis Be antigen), HIV antigen, HIV combined, H. pylori, β-CrossLaps, osteocalcin, PTH (parathyroid hormone), IgE, digoxin, digitoxin, AFP (α-fetoprotein), CEA (carcinoembryonic antigen), PSA (prostate specific antigen), free PSA, CA (cancer antigen) 19-9, CA 12-5, CA 72-4, cyfra 21-1, NSE (neuron specific enolase), S 100, P1NP (procollagen type 1 N-propeptide), PAPP-A (pregnancy-associated plasma protein-A), Lp-PLA2 (lipoprotein-associated phospholipase A2), sCD40L (soluble CD40 Ligand), IL 18, and Survivin.

Other exemplary, possibly monoclonal, antibodies include anti-TPO (antithyroid peroxidase antibody), anti-HBc (Hepatitis Bc antigen), anti-HBc/IgM, anti-HAV (hepatitis A virus), anti-HAV/IgM, anti-HCV (hepatitis C virus), anti-HIV, anti-HIV p-24, anti-rubella IgG, anti-rubella IgM, anti-toxoplasmosis IgG, anti-toxoplasmosis IgM, anti-CMV (cytomegalovirus) IgG, anti-CMV IgM, anti-HGV (hepatitis G virus), and anti-HTLV (human T-lymphotropic virus).

Examples of binding partners include binding proteins, for example, vitamin B12 binding protein, DNA binding proteins such as the superclasses of basic domains, zinc-coordinating DNA binding domains, Helix-turn-helix, beta scaffold factors with minor groove contacts, and other transcription factors that are not antibodies.

The term “labeled binding partner,” as used herein, means a binding partner that is labeled with an atom, moiety, functional group, molecule, or collection of molecules capable of generating, modifying or modulating a detectable signal. For example, in a radiochemical assay, the labeled binding partner may be labeled with a radioactive isotope of iodine. Alternatively, the labeled binding partner antibody may be labeled with an enzyme—e.g., horseradish peroxidase—that can be used in a colorimetric assay. The labeled binding partner may also be labeled with a time-resolved fluorescence reporter or a fluorescence resonance energy transfer (FRET) reporter. Exemplary reporters are disclosed in Hemmila, et al., J. Biochem. Biophys. Methods, vol. 26, pp. 283-290 (1993); Kakabakos, et al., Clin. Chem., vol. 38, pp. 338-342 (1992); Xu, et al., Clin. Chem., pp. 2038-2043 (1992); Hemmila, et al., Scand. J. Clin. Lab. Invest., vol. 48, pp. 389-400 (1988); Bioluminescence and Chemiluminescence Proceedings of the 9th International Symposium 1996, J. W. Hastings, et al., Eds., Wiley, New York, 1996; Bioluminescence and Chemiluminescence Instruments and Applications, Knox Van Dyre, Ed., CRC Press, Boca Raton, 1985; I. Hemmila, Applications of Fluorescence in Immunoassays, Chemical Analysis, Volume 117, Wiley, N.Y., 1991; and Blackburn, et al., Clin. Chem., vol. 37, p. 1534 (1991).

Further examples of labeled binding partners include binding partners that are labeled with a moiety, functional group, or molecule that is useful for generating a signal in an electrochemiluminescent (ECL) assay. The ECL moiety may be any compound that can be induced to repeatedly emit electromagnetic radiation by direct exposure to an electrochemical energy source. Such moieties, functional groups, or molecules are disclosed in U.S. Pat. Nos. 5,962,218; 5,945,344; 5,935,779; 5,858,676; 5,846,485; 5,811,236; 5,804,400; 5,798,083; 5,779,976; 5,770,459; 5,746,974; 5,744,367; 5,731,147; 5,720,922; 5,716,781; 5,714,089; 5,705,402; 5,700,427; 5,686,244; 5,679,519; 5,643,713; 5,641,623; 5,632,956; 5,624,637; 5,610,075; 5,597,910; 5,591,581; 5,543,112; 5,466,416; 5,453,356; 5,310,687; 5,296,191; 5,247,243; 5,238,808; 5,221,605; 5,189,549; 5,147,806; 5,093,268; 5,068,088; 5,935,779; 5,061,445; and 6,808,939; Dong, L. et al., AnaL Biochem., vol. 236, pp. 344-347 (1996); Blohm, et al., Biomedical Products, vol. 21, No. 4:60 (1996); Jameison, et al., Anal. Chem., vol. 68, pp. 1298-1302 (1996); Kibbey, et al., Nature Biotechnology, vol. 14, no. 3, pp. 259-260 (1996); Yu, et al., Applied and Environmental Microbiology, vol. 62, no. 2, pp. 587-592 (1996); Williams, American Biotechnology, p. 26 (January, 1996); Darsley, et al., Biomedical Products, vol. 21, no. 1, p. 133 (January, 1996); Kobrynski, et al., Clinical and Diagnostic Laboratory Immunology, vol. 3, no. 1, pp. 42-46 (January 1996); Williams, IVD Technology, pp. 28-31 (November, 1995); Deaver, Nature, vol. 377, pp. 758-760 (Oct. 26, 1995); Yu, et al., BioMedical Products, vol. 20, no. 10, p. 20 (October, 1995); Kibbey, et al., BioMedical Products, vol. 20, no. 9, p. 116 (September, 1995); Schutzbank, et al., Journal of Clinical Microbiology, vol. 33, pp. 2036-2041 (August, 1995); Stern, et al., Clinical Biochemistry, vol. 28, pp. 470-472 (August, 1995); Carlowicz, Clinical Laboratory News, vol. 21, no. 8, pp. 1-2 (August 1995); Gatto-Menking, et al., Biosensors & Bioelectronics, vol. 10, pp. 501-507 (July, 1995); Yu, et al., Journal of Bioluminescence and Chemiluminescence, vol. 10, pp. 239-245 (1995); Van Gemen, et al., Journal of Virology Methods, vol. 49, pp. 157-168 (1994); Yang, et al., Bio/Technology, vol. 12, pp. 193-194 (1994); Kenten, et al., Clinical Chemistry, vol. 38, pp. 873-879 (1992); Kenten, Non-radioactive Labeling and Detection of Biomolecules, Kessler, Ed., Springer, Berlin, pp. 175-179 (1992); Gudibande, et al., Journal of Molecular and Cellular Probes, vol. 6, pp. 495-503 (1992); Kenten, et al., Clinical Chemistry, vol. 37, pp. 1626-1632 (1991); and Blackburn, et al., Clinical Chemistry, vol. 37, pp. 1534-1539 (1991); and Electrogenerated Chemiluminescence, Bard, Editor, Marcel Dekker, (2004).

The term “analyte,” as used herein, means any molecule, or aggregate of molecules, including a cell or a cellular component of a virus, found in a sample. Examples of analytes to which the binding partner can specifically bind include bacterial toxins, viruses, bacteria, proteins, hormones, DNA, RNA, drugs, antibiotics, nerve toxins, and metabolites thereof. Also included in the scope of the term “analyte” are fragments of any molecule found in a sample. An analyte may be an organic compound, an organometallic compound or an inorganic compound. An analyte may be a nucleic acid (e.g., DNA, RNA, a plasmid, a vector, or an oligonucleotide), a protein (e.g., an antibody, an antigen, a receptor, a receptor ligand, or a peptide), a lipoprotein, a glycoprotein, a ribo- or deoxyribonucleoprotein, a peptide, a polysaccharide, a lipopolysaccharide, a lipid, a fatty acid, a vitamin, an amino acid, a pharmaceutical compound (e.g., tranquilizers, barbiturates, opiates, alcohols, tricyclic antidepressants, benzodiazepines, anti-virals, anti-fungals, antibiotics, steroids, cardiac glycosides, or a metabolite of any of the preceding), a hormone, a growth factor, an enzyme, a coenzyme, an apoenzyme, a hapten, a lectin, a substrate, a cellular metabolite, a cellular component or organelle (e.g., a membrane, a cell wall, a ribosome, a chromosome, a mitochondria, or a cytoskeleton component). Also included in the definition are toxins, pesticide, herbicides, and environmental pollutants. The definition further includes complexes comprising one or more of any of the examples set forth within this definition.

Further examples of analytes include bacterial pathogens such as Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psiffaci; Clostridium botulinum; Clostridium botulinum; Clostridium perfringens; Coccidioides immitis; Coccidioides posadasii; Cowdria ruminantium (Heartwater); Coxiella burnetii; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EPEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerospora philippinensis; Phakopsora pachyrhizi; Plesiomonas shigelloides; Ralstonia solanacearum race 3, biovar 2; Rickeffsia prowazekii; Rickettsia rickettsii; Salmonella spp.; Schlerophthora rayssiae var zeae; Shigella spp.; Staphylococcus aureus; Sfreptococcus; Synchytrium endobioticum; Vibrio cholerae non-O1; Vibrio cholerae O1; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

Further examples of analytes include viruses such as African horse sickness virus; African swine fever virus; Akabane virus; Avian influenza virus (highly pathogenic); Bhanja virus; Blue tongue virus (Exotic); Camel pox virus; Cercopithecine herpesvirus 1; Chikungunya virus; Classical swine fever virus; Coronavirus (SARS); Crimean-Congo hemorrhagic fever virus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (Exotic); Marburg virus; Mayaro virus; Menangle virus; Monkeypox virus; Mucambo virus; Newcastle disease virus (VVND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fever virus; Rinderpest virus; Rotavirus; Semliki Forest virus; Sheep pox virus; South American hemorrhagic fever viruses such as Flexal, Guanarito, Junin, Machupo, and Sabia; Spondweni virus; Swine vesicular disease virus; Tick-borne encephalitis complex (flavi) viruses such as Central European tick-borne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicular stomatitis virus (Exotic); Wesselbron virus; West Nile virus; Yellow fever virus; and South American hemorrhagic fever viruses such as Junin, Machupo, Sabia, Flexal, and Guanarito.

Further examples of analytes include toxins such as Abrin; Aflatoxins; Botulinum neurotoxin; Ciguatera toxins; Clostridium perfringens epsilon toxin; Conotoxins; Diacetoxyscirpenol; Diphtheria toxin; Grayanotoxin; Mushroom toxins such as amanitins, gyromitrin, and orellanine; Phytohaemagglutinin; Pyrrolizidine alkaloids; Ricin; Saxitoxin; Shellfish toxins (paralytic, diarrheic, neurotoxic, or amnesic) as saxitoxin, akadaic acid, dinophysis toxins, pectenotoxins, yessotoxins, brevetoxins, and domoic acid; Shigatoxins; Shiga-like ribosome inactivating proteins; Snake toxins; Staphylococcal enterotoxins; T-2 toxin; and Tetrodotoxin.

Further examples of analytes include prion proteins such as Bovine spongiform encephalopathy agent.

Further examples of analytes include parasitic protozoa and worms, such as: Acanthamoeba and other free-living amoebae; Anisakis sp. and other related worms Ascaris lumbricoides and Trichuris trichiura; Cryptosporidium parvum; Cyclospora cayetanensis; Diphyllobothrium spp.; Entamoeba histolytica; Eustrongylides sp.; Giardia lamblia; Nanophyetus spp.; Shistosoma spp.; Toxoplasma gondii; and Trichinella. Further examples of analytes include allergens such as plant pollen and wheat gluten.

Further examples of analytes include fungi such as: Aspergillus spp.; Blastomyces dermatitidis; Candida; Coccidioides immitis; Coccidioides posadasii; Cryptococcus neoformans; Histoplasma capsulatum; Maize rust; Rice blast; Rice brown spot disease; Rye blast; Sporothrix schenckii; and wheat fungus.

Further examples of analytes include genetic elements, recombinant nucleic acids, and recombinant organisms, such as:

(1) nucleic acids (synthetic or naturally derived, contiguous or fragmented, in host chromosomes or in expression vectors) that can encode infectious and/or replication competent forms of any of the select agents;

(2) nucleic acids (synthetic or naturally derived) that encode the functional form(s) of any of the toxins listed if the nucleic acids:

(i) are in a vector or host chromosome;

(ii) can be expressed in vivo or in vitro; or

(iii) are in a vector or host chromosome and can be expressed in vivo or in vitro;

(3) nucleic acid-protein complexes that are locations of cellular regulatory events:

(i) viral nucleic acid-protein complexes that are precursors to viral replication;

(ii) RNA-protein complexes that modify RNA structure and regulate protein transcription events; or

(iil) Nucleic acid-protein complexes that are regulated by hormones or secondary cell signaling molecules; or

(4) viruses, bacteria, fungi, and toxins that have been genetically modified.

Further examples of analytes include immune response molecules to the above-mentioned analyte examples such as IgA, IgD, IgE, IgG, and IgM.

The term “analog of the analyte,” as used herein, refers to a substance that competes with the analyte of interest for binding to a binding partner. An analog of the analyte may be a known amount of the analyte of interest itself that is added to compete for binding to a specific binding partner with analyte of interest present in a sample. Examples of analogs of the analyte include azidothymidine (AZT), an analog of a nucleotide that binds to HIV reverse transcriptase, puromycin, an analog of the terminal aminoacyl-adenosine part of aminoacyl-tRNA, and methotrexate, an analog of tetrahydrofolate. Other analogs may be derivatives of the analyte of interest.

The term “labeled analog of the analyte,” as used herein, is defined analogously to the term “labeled binding partner”, wherein the binding partner is substituted with analog of the analyte.

The term “ECL moiety” refers to any compound that can be induced to repeatedly emit electromagnetic radiation by exposure to an electrochemical energy source. Representative ECL moieties are described in Electrogenerated Chemiluminescence, Bard, Editor, Marcel Dekker, (2004); Knight, A and Greenway, G. Analyst 119:879-890 1994; and in U.S. Pat. Nos. 5,221,605; 5,591,581; 5,858,676; and 6,808,939. Preparation of primers comprising ECL moieties is well known in the art, as described, for example, in U.S. Pat. No. 6,174,709. Some ECL moieties emit electromagnetic radiation in the visible spectrum while others might emit other types of electromagnetic radiation, such as infrared or ultraviolet light, X-rays, and microwaves. Use of the terms “electrochemiluminescence”, “electrochemiluminescent”, “electrochemiluminesce”, “luminescence”, “luminescent” and “luminesce” in connection with the embodiments disclosed herein does not require that the emission be light. The emission may be forms of electromagnetic radiation other than light.

ECL moieties can be transition metals. For example, the ECL moiety can comprise a metal-containing organic compound wherein the metal may be chosen from, for example, ruthenium, osmium, rhenium, iridium, rhodium, platinum, palladium, molybdenum, and technetium. For example, the metal can be ruthenium or osmium. For example, the ECL moiety can be a ruthenium chelate or an osmium chelate. For example, the ECL moiety can comprise bis(2,2′-bipyridyl)ruthenium(II) and tris(2,2′-bipyridyl)ruthenium(II). For example, the ECL moiety can be ruthenium (II) tris bipyridine ([Ru(bpy)₃]²⁺). The metal can also be chosen, for example, from rare earth metals, including but not limited to cerium, dysprosium, erbium, europium, gadolinium, holmium, lanthanum, lutetium, neodymium, praseodymium, promethium, terbium, thulium, and ytterbium. For example, the metal can be cerium, europium, terbium, or ytterbium.

Metal-containing ECL moieties can have the formula
M(P)ₘ(L1)ₙ(L2)ₒ(L3)ₚ(L4)_{q}(L5)ᵣ(L6)ₛ

wherein M is a metal; P is a polydentate ligand of M; L1, L2, L3, L4, L5 and L6 are ligands of M, each of which can be the same as, or different from, each other; m is an integer equal to or greater than 1; each of n, o, p, q, r and s is an integer equal to or greater than zero; and P, L1, L2, L3, L4, L5 and L6 are of such composition and number that the ECL moiety can be induced to emit electromagnetic radiation and the total number of bonds to M provided by the ligands of M equals the coordination number of M. For example, M may be chosen from ruthenium or osmium.

Some examples of the ECL moiety can have one polydentate ligand of M. The ECL moiety can also have more than one polydentate ligand. In examples comprising more than one polydentate ligand of M, the polydentate ligands can be the same or different. Polydentate ligands can be aromatic or aliphatic ligands. Suitable aromatic polydentate ligands can be aromatic heterocyclic ligands and can be nitrogen-containing, such as, for example, bipyridyl, bipyrazyl, terpyridyl, 1,10-phenanthroline, and porphyrins.

Suitable polydentate ligands can be unsubstituted, or substituted by any of a large number of substituents known to the art. Suitable substituents include, but are not limited to, alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, maleimide sulfur-containing groups, phosphorus-containing groups, and the carboxylate ester of N-hydroxysuccinimide.

In some embodiments, at least one of L1, L2, L3, L4, L5 and L6 can be a polydentate aromatic heterocyclic ligand. In various embodiments, at least one of these polydentate aromatic heterocyclic ligands can contain nitrogen. Suitable polydentate ligands can be, but are not limited to, bipyridyl, bipyrazyl, terpyridyl, 1,10-phenanthroline, a porphyrin, substituted bipyridyl, substituted bipyrazyl, substituted terpyridyl, substituted 1,10-phenanthroline or a substituted porphyrin. These substituted polydentate ligands can be substituted with an alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, maleimide a sulfur-containing group, a phosphorus-containing group or the carboxylate ester of N-hydroxysuccinimide.

Some ECL moieties can contain two bidentate ligands, each of which can be bipyridyl, bipyrazyl, terpyridyl, 1,10-phenanthroline, substituted bipyridyl, substituted bipyrazyl, substituted terpyridyl or substituted 1,10-phenanthroline.

Some ECL moieties can contain three bidentate ligands, each of which can be bipyridyl, bipyrazyl, terpyridyl, 1,10-phenanthroline, substituted bipyridyl, substituted bipyrazyl, substituted terpyridyl or substituted 1,10-phenanthroline. For example, the ECL moiety can comprise ruthenium, two bidentate bipyridyl ligands, and one substituted bidentate bipyridyl ligand. For example, the ECL moiety can contain a tetradentate ligand such as a porphyrin or substituted porphyrin.

In some embodiments, the ECL moiety can have one or more monodentate ligands, a wide variety of which are known to the art. Suitable monodentate ligands can be, for example, carbon monoxide, cyanides, isocyanides, halides, and aliphatic, aromatic and heterocyclic phosphines, amines, stibines, and arsines.

In some embodiments, one or more of the ligands of M can be attached to additional chemical labels, such as, for example, radioactive isotopes, fluorescent components, or additional luminescent ruthenium- or osmium-containing centers.

For example, the ECL moiety can be tris(2,2′-bipyridyl)ruthenium(II) tetrakis(pentafluorophenyl)borate. For example, the ECL moiety can be bis[(4,4′-carbomethoxy)-2,2′-bipyridine] 2-[3-(4-methyl-2,2′-bipyridine-4-yl)propyl]-1,3-dioxolane ruthenium (II). For example, the ECL moiety can be bis(2,2′bipyridine) [4-(butan-1-al)-4′-methyl-2,2′-bipyridine]ruthenium (II). For example, the ECL moiety can be bis(2,2′-bipyridine) [4-(4′-methyl-2,2′-bipyridine-4′-yl)-butyric acid]ruthenium (II). For example, the ECL moiety can be (2,2′-bipyridine)[cis-bis(1,2-diphenylphosphino)ethylene]{2-[3-(4-methyl-2,2′-bipyridine-4′-yl)propyl]-1,3-dioxolane}osmium (II). For example, the ECL moiety can be bis(2,2′-bipyridine) [4-(4′-methyl-2,2′-bipyridine)-butylamine]ruthenium (II). For example, the ECL moiety can be bis(2,2′-bipyridine) [1-bromo-4(4′-methyl-2,2′-bipyridine-4-yl)butane]ruthenium (II). For example, the ECL moiety can be bis(2,2′-bipyridine)maleimidohexanoic acid, 4-methyl-2,2′-bipyridine-4′-butylamide ruthenium (II).

In some embodiments, an assay-performance-substance is used, wherein the assay-performance-substance comprises an ECL moiety and a labeled binding partner for an analyte or a labeled analog of the analyte.

In some embodiments, the assay-performance-substance comprises an ECL moiety.

In some embodiments, the ECL moiety comprises a metal ion. In further embodiments, the metal ion may be chosen from osmium and ruthenium.

In some embodiments, the ECL moiety comprises a derivative of trisbipyridyl ruthenium (II) [Ru(bpy)₃²⁺]. For example, the ECL moiety can be [Ru(sulfo-bpy)₂bpy]²⁺whose structure is provided by: 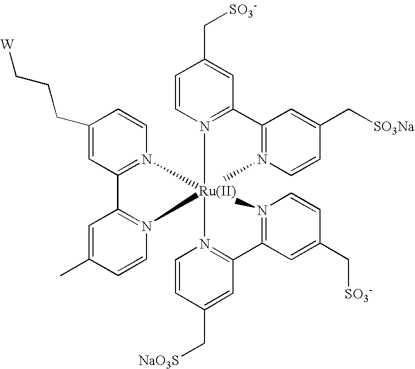
wherein W is a functional group attached to the ECL moiety capable of reacting with a biological material, binding reagent, enzyme substrate or other assay reagent, thereby forming a covalent linkage. The covalent linkage may be chosen from a NHS ester, an activated carboxyl, an amino group, a hydroxyl group, a carboxyl group, a hydrazide, a maleimide, and a phosphoramidite.

In some embodiments, the ECL moiety does not comprise a metal. Such non-metal ECL moieties may be chosen from rubrene and 9,10-diphenylanthracene.

The tem “ECL coreactant,” as used herein, pertains to a chemical compound that either by itself or via its electrochemical reduction oxidation product(s), plays a role in the ECL reaction sequence.

Often ECL coreactants can permit the use of simpler means for generating ECL (e.g., the use of only half of the double-step oxidation-reduction cycle) and/or improved ECL intensity. In some embodiments, coreactants can be chemical compounds that, upon electrochemical oxidation/reduction, yield, either directly or upon further reaction, strong oxidizing or reducing species in solution. A coreactant can be peroxodisulfate (i.e., S₂O₈²⁻, persulfate) that is irreversibly electro-reduced to form oxidizing SO₄.—ions. The coreactant can also be oxalate (i.e., C₂O₄²⁻) that is irreversibly electro-oxidized to form reducing CO₂.—ions. A class of coreactants that can act as reducing agents is amines or compounds containing amine groups, including, for example, tri-n-propylamine (i.e., N(CH₂CH₂CH₂)₃, TPA). The amine coreactants may be chosen from primary amines, secondary amines, and tertiary amines.

In some embodiments, the biological detection system comprises an ECL coreactant. In some embodiments, the multi-well reagent container comprises an ECL coreactant. These coreactants can be, for example, tertiary or secondary amines or other coreactants described herein.

In some embodiments, the ECL coreactant comprises a tertiary amine comprising a hydrophilic functional group.

In some embodiments, the ECL coreactant is an amine having a structure
NR¹R²R³
wherein R¹, R² and R³ are each C₁₋₁₀aliphatic groups, and wherein at least one of the C₁₋₁₀ aliphatic groups is substituted with at least one hydrophilic functional group. In some embodiments, the hydrophilic functional group may be a charged group, for example, a negatively charged group. Hydrophilic functional groups may be chosen from hydroxyl, hydroxycarbonyl, amino, aminocarbonyl, amidine, imino, cyano, nitro, nitrate, sulfate, sulfonate, phosphate, phosphonate, silicate, carboxylate, borate (B(OH)₃), guanidinium, carbamide, carbamate, carbonate, sulfamide, silyl, siloxy, and amide groups.

In some embodiments, the ECL coreactant may have the structure
(n-propyl)₂N(CH₂)ₙ₁R*
wherein n1 is an integer from 1 to 10; and R* is a hydrophilic functional group, as defined herein. In some embodiments, n1 is 2, 3, and 4.

In some embodiments, the ECL coreactant may be a compound having the formula 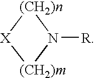
wherein X is chosen from —(CH₂)—, —(CHR¹¹)—, —(CR¹¹R¹²)—, a heteroatom, and —N(R¹¹)—;

R is a C₁₋₁₀ aliphatic group substituted with at least one hydrophilic functional group; each of R¹¹ and R¹² is, independently, a C₁₋₁₀ aliphatic group optionally substituted with at least one hydrophilic functional group; and

n and m are, independently, integers ranging from 1 to 10.

In some embodiments, the heteroatom can be, for example, —O— or —S—.

In some embodiments, n may be chosen from 2, 3, and 4. In some embodiments, m may be chosen from 2, 3, and 4.

In some embodiments, R¹¹ is a C₁₋₄ aliphatic group.

In some embodiments, R is a C₁₋₄ aliphatic group substituted with at least one hydrophilic functional group.

When X is —N(R¹¹), R¹¹ can be, for example, (CH₂)ₙ₃—R¹³, wherein n3 is an integer ranging from 3 to 20 or ranging from 3 to 10, and R¹³ is H, an aliphatic group, or a hydrophilic functional group. In further embodiments, n3 may be chosen from 3 and 4.

In some embodiments, R is —(CH₂)ₙ₂—R¹², wherein n2 is an integer ranging from 3 to 20 or ranging from 3 to 10. In further embodiments, n2 may be chosen from 3, 4, and 5.

In some embodiments, R¹² may be a hydrophilic functional group. In some embodiments, R¹² may be a carboxylate or sulfonate.

The use of ECL coreactants having hydrophilic functional groups (and, in particular, ECL coreactants that are zwitterionic at neutral pH) has a variety of advantages that are unrelated to their ability to act as ECL coreactants. These species tend to be highly water soluble and to have low vapor pressure. Thus, it is possible to produce highly concentrated stock solutions that may be diluted as necessary for use. It is also possible to prepare dried reagents comprising the ECL coreactants without uncertainty due to loss of ECL coreactant in the vapor phase. Furthermore, when present in a dry composition, these ECL coreactants resolubilize quickly in a minimum of volume.

Coreactants include, but are not limited to, lincomycin; clindamycin-2-phosphate; erythromycin; 1-methylpyrrolidone; diphenidol; atropine; trazodone; hydroflumethiazide; hydrochlorothiazide; clindamycin; tetracycline; streptomycin; gentamicin; reserpine; trimethylamine; tri-n-butylphosphine; piperidine; N,N-dimethylaniline; pheniramine; bromopheniramine; chloropheniramine; diphenylhydramine; 2-dimethylaminopyridine; pyrilamine; 2-benzylaminopyridine; leucine; valine; glutamic acid; phenylalanine; alanine; arginine; histidine; cysteine; tryptophan; tyrosine; hydroxyproline; asparagine; methionine; threonine; serine; cyclothiazide; trichlormethiazide; 1,3-diaminopropane; piperazine, chlorothiazide; hydrazinothalazine; barbituric acid; persulfate; penicillin; 1-piperidinyl ethanol; 1,4-diaminobutane; 1,5-diaminopentane; 1,6-diaminohexane; ethylenediamine; benzenesulfonamide; tetramethylsulfone; ethylamine; di-ethylamine; tri-ethylamine; tri-iso-propylamine; di-n-propylamine; di-iso-propylamine; di-n-butylamine; tri-n-butylamine; tri-iso-butylamine; bi-iso-butylamine; s-butylamine; t-butylamine; di-n-pentylamine; tri-n-pentylamine; n-hexylamine; hydrazine sulfate; glucose; n-methylacetamide; phosphonoacetic acid; and/or salts thereof.

ECL coreactants include, but are not limited to, 1-ethylpiperidine; 2,2-Bis(hydroxymethyl)-2,2′,2″-nitrilotriethanol (BIS-TRIS); 1,3-bis[tris(hydroxymethyl)methylamino]propane (bis-Tris propane) (BIS-TRIS propane); 2-Morpholinoethanesulfonic acid (MES); 3-(N-Morpholino)propanesulfonic acid (MOPS); 3-Morpholino-2-hydroxypropanesulfonic acid (MOPSO); 4-(2-Hydroxyethyl)piperazine-1-(2-hydroxypropanesulfonic acid) (HEPPSO); 4-(2-Hydroxyethyl)piperazine-1-propanesulfonic acid (EPPS); 4-(N-Morpholino)butanesulfonic acid (MOBS);N,N-Bis(2-hydroxyethyl)glycine (BICINE); DAB-AM-16, Polypropylenimine hexadecaamine Dendrimer (DAB-AM-16); DAB-AM-32, Polypropylenimine dotriacontaamine Dendrimer (DAB-AM-32); DAB-AM-4, Polypropylenimine tetraamine Dendrimer (DAB-AM-4); DAB-AM-64, Polypropylenimine tetrahexacontaamine Dendrimer; DAB-AM-8, Polypropylenimine octaamine Dendrimer (DAB-AM-8); di-ethylamine; dihydronicotinamide adenine dinucleotide (NADH); di-iso-butylamine; di-iso-propylamine; di-n-butylamine; di-n-pentylamine; di-n-propylamine; di-n-propylamine; ethylenediamine tetraacetic acid (EDTA); Glycyl-glycine (Gly-Gly); N-(2-Acetamido)iminodiacetic acid (ADA); N-(2-Hydroxyethyl)piperazine-N′-(2-ethanesulfonic acid) (HEPES); N-(2-Hydroxyethyl)piperazine-N′-(4-butanesulfonic acid) (HEPBS); N,N-Bis(2-hydroxyethyl)-3-amino-2-hydroxypropanesulfonic acid (DIPSO); N,N-Bis(2-hydroxyethyl)taurine (BES); N-ethylmorpholine; oxalic acid; Piperazine-1,4-bis(2-hydroxypropanesulfonic acid) (POPSO); s-butylamine; sparteine; t-butylamine; triethanolamine; tri-ethylamine; tri-iso-butylamine; tri-iso-propylamine; tri-n-butylamine; tri-n-butylamine; tri-n-pentylamine; N,N,N′,N′-Tetrapropyl-1,3-diaminopropane; oxalate; peroxodisulfate; piperazine-1,4-bis(2-ethanesulfonic acid) (PIPES); tri-n-propylamine; 3-dimethylamino-1-propanol; 3-dimethylamino-2-propanol; 1,3-Bis(dimethylamino)-2-propanol; 1,3-Bis(diethylamino)-2-propanol; 1,3-Bis(dipropylamino)-2-propanol; N-tris(hydroxymethyl)methyl-2-aminoethane sulfonic acid (TES); piperazine-N,N′-bis-3-propanesulfonic acid (PIPPS); piperazine-N,N′-bis-4-butanesulfonic acid (PIPBS); 1,6-diaminohexane-N,N,N′,N′-tetraacetic acid; 4-(di-n-propylamino)-butanesulfonic acid; 4-[bis-(2-hydroxyethane)-amino]-butanesulfonic acid; azepane-N-(3-propanesulfonic acid); N,N-bis propyl-N-4-aminobutanesulfonic acid; piperazine-N,N′-bis-3-methylpropanoate; piperazine-N-2-hydroxyethane-N′-3-methylpropanoate; piperidine-N-(3-propanesulfonic acid); piperidine-N-(3-propionic acid) (PPA); 3-(di-n-propylamino)-propanesulfonic acid; and/or salts thereof.

In some embodiments, the ECL coreactant may be chosen from piperazine-1,4-bis(2-ethanesulfonic acid) (PIPES), tri-n-propylamine, N,N,N′,N′-Tetrapropyl-1,3-diaminopropane, 1,3-Bis(dipropylamino)-2-propanol, and salts and mixtures thereof. In some embodiments, the ECL coreactant may be chosen from oxalate or tri-n-propylamine.

The term “positive control/calibrator,” as used herein, refers to a known amount of analyte or an analog of the analyte. In some embodiments, positive control/calibrators further comprise a sample matrix similar to that a sample is expected to have. Positive control/calibrators can be used to assess the proper operation of the instrumentation and/or the sample measurement. Positive control/calibrators alone or in combination with negative control/calibrators can be used as a reference to compare the signal level of the test sample with the signal level of the reference. Positive control/calibrators alone or in combination with negative control/calibrators can also be used along with a mathematical function to relate signal levels with analyte concentrations, one use of which is to convert a signal measurement from a sample to an analyte concentration. The term “positive control/calibrator” encompasses the common definition of both positive control and positive calibrator.

The term “negative control/calibrator,” as used herein, refers to a sample matrix similar to that a sample is expected to have. Negative control/calibrators can be used to assess the proper operation of the instrumentation and/or the sample measurement. Negative control/calibrators can be used alone or in conjunction with positive control/calibrators as a reference to compare the signal level of the test sample with the signal level of the reference. Negative control/calibrators alone or in conjunction with positive control/calibrators can also be used along with a mathematical function to relate signal levels with analyte concentrations, one use of which is to convert a signal measurement from a sample to an analyte concentration. The term “negative control/calibrator” encompasses the common definition of both negative control and negative calibrator.

The term “control/calibrator,” as used herein, refers to either a positive control/calibrator or a negative control/calibrator.

The term “assay control/calibrator,” as used herein, refers to reagents used (a) to confirm successful measurement of a sample or (b) to convert a measured signal from a sample into a concentration of the tested analyte. In certain embodiments, an assay control/calibrator can comprise a positive control/calibrator and the reagents used for a binding assay in order to simulate measurements from a sample that contains the analyte. In certain embodiments, an assay control/calibrator can comprise a negative control/calibrator and the reagents used for a binding assay in order to simulate measurements from a sample that contains the analyte.

The term “sample,” as used herein, comprises liquids that may contain the analyte. The term “liquid,” as used herein comprises—in addition to the more traditional definition of liquid—colloids, suspensions, slurries, and dispersions of particles (including beads) in a liquid wherein the particles have a sedimentation rate due to earth's gravity of less than or equal to about 1 mm/s. The sample can be drawn from any source upon which analysis is desired. For example, the sample can arise from body or other biological fluid, such as blood, plasma, serum, milk, semen, amniotic fluid, cerebral spinal fluid, sputum, bronchoalveolar lavage, tears, urine, saliva, or stool. Alternatively, the sample can be a water sample obtained from a body of water, such as lake or river, or it may be from a source of drinking water, such as a tap, aquifer, reservoir, or water purification system. The sample can also be prepared by dissolving or suspending a sample in a liquid, such as water or an aqueous buffer. The sample source can be a surface swab. For example, a surface can be swabbed, and the swab washed by a liquid, thereby transferring an analyte from the surface into the liquid. The sample source can be air. For example, the air can be filtered, and the filter washed by a liquid, thereby transferring an analyte from the air into the liquid.

The term “sample matrix,” as used herein, refers to everything in the sample with the exception of the analyte.

The term “environmental matrix”, as used herein, refers to components of the sample matrix derived from the environment from which the sample is collected.

The term “magnetic field source,” as used herein, includes permanent magnets and electromagnets, which are separate, individual entities with defined N-S magnetic poles. A dipole magnet comprises one magnetic field source.

The term “sandwich magnet,” as used herein, refers to magnets comprising two or more magnetic field sources configured such that their opposing magnetic fields overlap or are coerced. This can be accomplished by placing opposing poles (N-N or S-S) in closer proximity to each other than the attracting poles (N-S) of the magnetic fields sources. For example, two dipole magnets arranged in an N-S-S-N or a S-N-N-S configuration would form a sandwich magnet.

The term “channel magnet,” as used herein, refers to a single magnetic field source bonded to a highly magnetizable material in the form of a U-shaped channel. In such a configuration, the magnetizable material becomes an extension of the magnetic pole to which it is bound.

The term “assay well,” as used herein, refers to a well in a multi-well reagent container that comprises binding reagents specific for an analyte of interest.

As used herein, the term “support,” refers to any of the ways for immobilizing binding partners that are known in the art, such as membranes, beads, particles, electrodes, or even the walls or surfaces of a container. The support may comprise any material on which the binding partner is conventionally immobilized, such as nitrocellulose, polystyrene, polypropylene, polyvinyl chloride, EVA, glass, carbon, glassy carbon, carbon black, carbon nanotubes or fibrils, platinum, palladium, gold, silver, silver chloride, iridium, or rhodium. In one embodiment, the support is a bead, such as a polystyrene bead or a magnetizable bead. Beads are inanimate. As used herein, the term “magnetizable bead” encompasses magnetic, paramagnetic, and superparamagnetic beads. In some embodiments, the support is a microcentrifuge tube or at least one well of a multiwell plate. Magnetizable beads useful in this invention include those with diameters ranging from 0.09 μm to 10 μm; from 0.4 μm to 3 μm; and from 0.9 μm to 3 μm.

The term “binding reagents,” as used herein, comprise a binding partner for an analyte of interest. Binding reagents optionally comprise a labeled binding partner for an analyte of interest and/or a labeled analog of the analyte. Binding reagents optionally comprise a support. Binding reagents optionally comprise a magnetizable bead. Binding reagents optionally comprise buffers, salts, cryoprotectants, surfactants, blocking agents, and other materials as well known in the art.

The biological detection system that is the subject of this invention can employ many assay formats that involve binding reactions: for example those described in U.S. Pat. No. 6,078,782, and in The Immunoassay Handbook, 3rd Edition, Wild, Editor, Stockton Press (2005) and Principles and Practice of Immunoassay, Price and Newman, Editors, Stockton Press (1997). Example formats include sandwich assays wherein a labeled binding partner specific for an analyte of interest and a second binding partner, specific for the same analyte, attached to a support can be used to link the label to the support in the presence of the analyte. Competitive formats using either a labeled binding partner or a labeled analyte or analog of the analyte are also contemplated. In some embodiments, the support, labeled species, and optional second binding partner are stored separately. The order and timing between the additions of these binding reagents vary, as known in the art. In some embodiments, the support, labeled species, and optional second binding partner are stored together—simplifying the steps required by the detection system to measure an analyte. In some embodiments, only the sample is required to be added to the binding reagents to form the complex between the support and the label that is to be measured.

II. Detection System

The present invention relates to a detection system used to measure one or more analytes of interest in a sample using binding reactions. In some embodiments, the detection system comprises a flow cell, a detector, at least one holder for a multi-well reagent container and a sample container, at least one multi-well reagent container having a binding partner for a binding reaction, a probe, a pump, and a liquid-level detector, where the flow cell and the probe are fluidically connected. In some embodiments, the detection system comprises a flow cell, a detector, at least one holder for a multi-well reagent container and a sample container, a probe, a pump, and 2 or more magnetic capture zones, where the flow cell, magnetic capture zones, and the probe are fluidically connected.

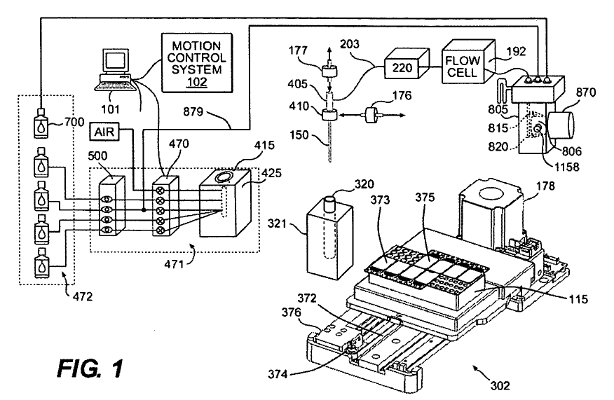

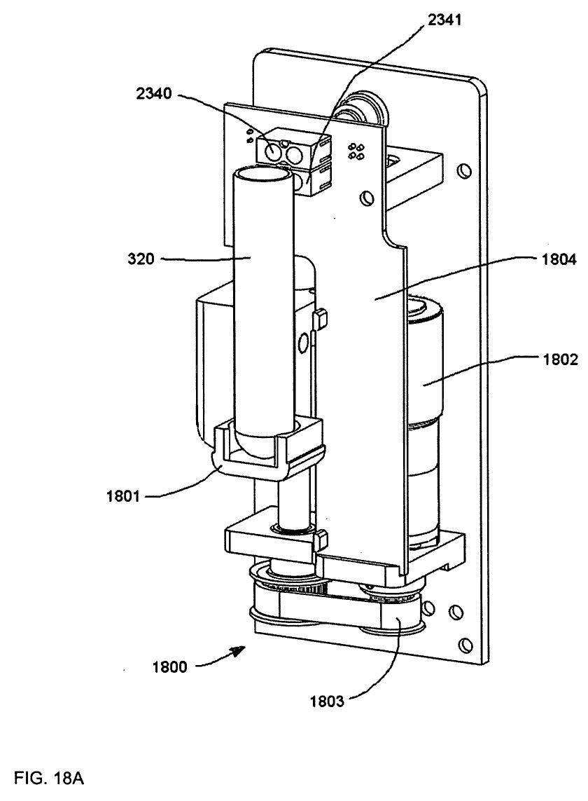

FIG. 1 and FIG. 15 are schematic representations of exemplary detection systems used to measure one or more analytes of interest possibly present in a sample through the use of binding reactions. A detector configured to detect a label used in binding reactions can be located in flow cell 192. The binding reagents for a plurality of measurements can be located in each multi-well reagent containers; three exemplary types are shown as 373, 375, and 550. These multi-well reagent containers can be held in the detection system using, for example, holder 115 or holder 1501 in carrier 302. The sample can be brought to the detection system in a sample container 320 and held in the system with sample container holder 321. Probe 150 and pump 870 can be configured to distribute a known amount of sample into an assay well of one or more of the multi-well reagent containers. A liquid level detector can be used to determine the presence of liquid and/or the liquid level in the sample container. In some embodiments, the liquid level detector can be used to detect the level of the liquid in individual wells of the multi-well reagent containers.

In some embodiments, the detection system can be operative to communicate information, such as test results or patient information, to one or more external devices, including but not limited to a pager, PDA, cell phone, wireless device, computer or printer. Data transmission can be accomplished through many techniques known in the art consistent with the principles of the present invention. Techniques for transmitting information to other devices that can be employed by the detection system include, but are not limited to, radio frequency transmission, near-infrared, TCP/IP, USB, FireWire® (IEEE 1394) (Apple; Cupertino Calif., USA) RS-232, RS-485, RS-422, Bluetooth®(Bluetooth Sig. Inc.; Bellevue, Wash., USA), and IEEE-802.11. Information can be transmitted to multiple individuals interested in the results of testing. The detection system can also employ encryption and/or data protection techniques to ensure the privacy of transmitted information. In addition to transmitting information to external devices, the detection system can also be adapted to receive information from external devices through the above-described techniques, as well as others known in the art.

A. Flow Cells

As depicted in FIG. 1 and FIG. 15, overall operation of the detection system may be conducted under control of a computer system 101. Sample analysis can occur in a flow cell 192, which can be a flow cell configured to measure radioactivity, optical absorbance, magnetic or magnetizable materials, light scattering, optical interference (i.e., interferometric measurements), refractive index changes, surface plasmon resonance, and/or luminescence (e.g., fluorescence, chemiluminescence and electrochemiluminescence). According to certain embodiments, flow cell 192 can be adapted for conducting luminescence measurements and can utilize a light detector to measure the luminescent emission. The light detector can comprise, for example, a photodiode (including PIN and avalanche photodiodes), a CCD, a CMOS sensor, a photomultiplier tube (PMT), or a channel multiplier tube (CMT). Exemplary electrochemiluminescence flow cells and methods for their use are disclosed in U.S. Pat. No. 6,200,531 and International Patent Application WO 99/58962. The detection system can be configured with an electrical energy source and an electrode, both suitable for initiating electrochemiluminescence. Exemplary electrodes comprise platinum, alloys of platinum and iridium, gold, and carbon. (See U.S. patent application Publication No. 2004/0090168). The operation of flow cell 192 can be controlled by computer system 101, which can also receive assay data from flow cell 192 and carry out data analysis.

B. Multi-well Reagent Container Holder

The multi-well reagent container holder 115 depicted in FIG. 1 can be adapted to hold at least one multi-well reagent container. As illustrated in FIG. 1, holder 115 can have a capacity to hold 6 nine-well containers 373 and 6 six-well containers 375-4 of each are shown. As illustrated in FIG. 15, holder 1501 can have a capacity to hold 2 multi-well reagent containers each having 5 assay wells. Holder 115 or holder 1501 can be part of a carrier 302 that can include a simple one degree of freedom device that translates holder 115 or holder 1501 linearly to allow a probe 150 to access each well of at least one of multi-well reagent containers 373, 375, or 550. Carrier 302 can optionally be adapted to have additional degrees of freedom in the vertical direction or in the plane of the container.

The system, however, is not limited to such a container alignment device and can utilize any system capable of enabling probe 150 to access each well of, for example, at least one of containers 373, 375, or 550. For example, a rotary system could be employed wherein containers 373, 375, or 550 are loaded on an arm that rotationally pivots about some point. The automated pipettor 405 shown in FIG. 1 and FIG. 15 can be capable of moving probe 150 in two dimensions within a Cartesian coordinate system through two independently controllable drive mechanisms 176, 177, which may comprise, for example, motors. Relative motion between probe 150 and holder 302 in a third direction, not parallel to the other two dimensions, may be affected through a third independently controllable drive mechanism 178. Drive mechanism 178 may translate holder 115 or holder 1501 via a belt 372 that travels between a drive mechanism 178 and a pulley 374. Drive mechanism 178 may also be used to agitate samples held by holder 115. A counterweight 376, attached to the opposite side of belt 372, may be used to reduce the vibrations of the rest of the system by moving in the opposite direction of holder 115 or holder 1501. The three directions of motion may be very close to mutually perpendicular, perhaps only having fabrication-related perturbations from perpendicularity, or may be distinctly non-perpendicular, perhaps due to the lack of a requirement to move over all points in a rectangular box. Alternatively, motion control systems based on alternative coordinate systems may be used (e.g., one dimensional, two dimensional, polar coordinates, etc.). Operation of the automation systems may be controlled by a motion control subsystem. As depicted, a motion control subsystem 102 may receive instructions from computerized system 101. Motion control subsystem 102 may be operative to convert the instructions into appropriate control signals that direct one or more of the automation systems to perform the necessary steps to carry out the instructions of computer system 101.

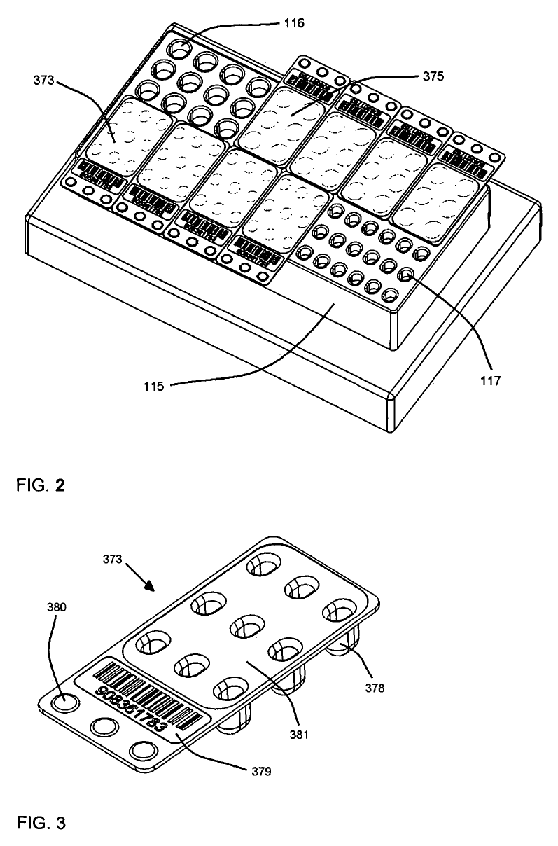

Turning now to FIG. 2, the multi-well reagent container holder 115 is shown in greater detail. As illustrated, holder 115 can have a capacity of 6 nine-well reagent containers 373 and 6 six-well reagent containers 375-4 of each are shown. Depressions 116 in holder 115 may be sized to form a close fit to the wells of container 375. Similarly, depressions 117 in holder 115 are sized to form a close fit to the wells of container 373. Other embodiments can include those that hold only one type of multi-well reagent container. Depressions 116, 117 may help align multi-well reagent containers 373, 375 to make them more accessible to pipettor 405. These depressions may also increase the contacted surface area between holder 115 and containers 373, 375. The increased surface area may be used as increased friction to prevent containers 373, 375 from moving relative to holder 115 during agitation. The increased surface area may be useful to decrease the thermal resistance between holder 115 and the wells of containers 373, 375.

Holder 115 may optionally be temperature-controlled, thereby regulating the temperature of the wells in containers 373, 375. To control the temperature of holder 115 to a temperature above ambient, a power resistor and a temperature sensor may be used. For example, a thin-film heating element such as a foil heater (Minco Corp, Minn.) may be used as the power resistor or both the power resistor and the temperature sensor. By adjusting the heating element composition, the resistance can be made to change minimally or substantially in relation to the temperature. Other temperature sensors that may be used include resistance temperature detectors (RTDs), thermocouples, and thermistors. To control the temperature of holder 115 to a temperature below ambient, a thermoelectric module utilizing the Peltier effect may be used. Electrical connection to holder 115 may be made, for example, through a flex-cable or through contacts that mate with matching contacts when carrier 302 positions holder 115 in an appropriate location. A similar temperature controller may be used to regulate the temperature where the measurement of label occurs, for example, in flow cell 192.

In some embodiments, holder 115 can lack depressions similar to depressions 116 or 117. In some embodiments, the multi-well reagent container can be smooth on the bottom, such as a flat-bottom 96 well microplate. The multi-well reagent container holder may hold and align the at least one container via the edges, flanges, or other alignment features on the container; for example, see holding mechanisms described in U.S. patent application Publication Nos. 2005/0250173 and 2004/009630. In some embodiments, holder 115 may be temperature controlled also.

Analogous to holder 115, holder 1501 can also be configured with depressions to assist in heat transfer to assay wells in container 550. Holder 1501 can be temperature controlled.

C. Fluid Transfer

The exemplary flow cell-based biological detection system may also comprise a fluid handling station for introducing one or more reagents and/or one or more samples, which may include gases and liquids. FIG. 1 depicts a fluid handling station 471 that may comprise flow control valves 470, reagent/gas detectors 500, and a fluid-handling manifold 425. These devices may be independent fixtures fluidically connected (e.g., through flexible tubing) or may be integrated into a single system (as indicated by the dashed line). In an alternative embodiment, the location of valves 470 and sensors 500 along the fluidic lines may be switched so that sensors 500 are between system reagents 472 and valves 470. System reagents 472 may be bottles, or they may be packaged as a unit along with a waste container 700 in a box comprising flexible bags. As the bags holding the system reagents empty, the space gained can be used to allow expansion of the waste bag, thereby reducing the overall volume occupied by the system reagents 472 and waste container 700.

The fluid-handling manifold 425 may include an aspiration chamber employing a face-sealing configuration using, for example, an o-ring 415 arranged on a sealing surface of manifold 425 that may be adapted to achieve a fluidic seal between manifold 425 and a sealing surface 410 of probe 150 (e.g., a collar, flange, or the like). As depicted, fluid-handling manifold sealing surface 410 can be located away from the reagent input lines (e.g., above the reagent lines' aspiration chamber entry points). Additionally, one or more of the reagent entry points can be positioned at predetermined heights within the aspiration chamber. For example, as depicted, the liquid reagent lines may be positioned beneath the gas reagent line to preclude contamination of the gas line. Reagent aspiration may be controlled by coordinating the selective actuation of one or more of reagent valves 470 with the proper positioning of pipettor 405 and activation of a pump 870 so as to draw reagents from selected system reagents 472. Reagent detectors 500 may be employed to determine the presence and/or absence of reagent (e.g., whether one or more of system reagents 472 are empty), to determine the presence and/or absence of gaseous reagents (e.g., when air is used to segment fluids as they are aspirated), to determine/confirm the aspirated volume of a particular reagent, etc.

In an alternate embodiment, as depicted in FIG. 15, the detection system may not use fluid handling station 471, or system reagents 472. In these embodiments, system reagents would instead be located in the multi-well reagent container 550.

As shown in FIG. 1, flow cell 192 may be connected to pipettor 405 through tubing 203. Tubing 203 may go through a prewash apperatus 220 before reaching flow cell 192. The prewash apperatus 220 and the flow cell 192 can both comprise magnets to form 2 magnetic capture zones to attract magnetizable beads located in the binding reagents. Prewash apperatus 220 and flow cell 192 can be sufficiently separated so as to have no operative magnetic influence on each other.

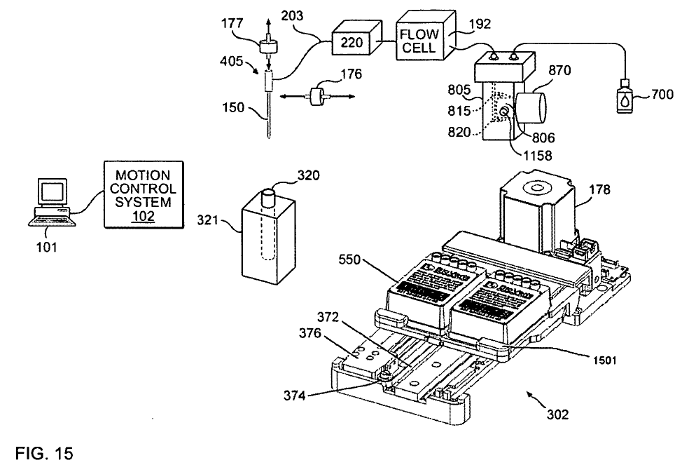

As shown in FIG. 1, sample container 320 may be held in a holder of a sample container 321. In further embodiments, holder 321 may hold a plurality of sample containers, and holder 321 may be located on carrier 302. As shown in FIG. 10, the sample container may be well 560 located in multi-well reagent container 550.

D. Pump

As shown in FIG. 1, the detection system may include a positive displacement pump 870. Pump 870 may be configured with a pump head manifold 805 that may be adapted to include a cleanout fluid path and plug 1158. Incorporation of cleanout path and plug 1158 allows the chamber of pump 870 (indicated by dashed lines) to be decontaminated in the event of failure of the piston of pump 870. Bubble and sediment purge pathways, as described in U.S. patent application Publication No. 2004/009638, improve the performance of pump 870. Pump 870 may aspirate and dispense from probe 150 and from waste container 700.

E. Temperature Controller

Holder 115 or holder 1501 and flow cell 192 may each have a temperature controller to regulate the temperature of the assay wells of containers 373, 375, or 550 and the temperature during the measurement process, respectively. The temperature controller may further regulate the temperature of the area surrounding the assay wells. Regulating the temperature of the assay wells of the multi-well reagent containers 373, 375, or 550 may be advantageous to, for example, (1) make the detection system less sensitive to variations is ambient temperature due to reaction rates (such as binding events) being temperature sensitive; and/or (2) reduce the time required for binding events to occur by operating at an elevated temperature (e.g., 34, 35, 36, 37 38, 39, 40, 41, 42, 43, 44, 45 or 65° C.). In some embodiments, multi-well reagent containers can be stored in the detection system to reduce the number of steps required by a user when presenting the detection system with a sample. Long-term reagent stability can also be affected by storage temperature. Consequently, when present, the temperature controller for containers 373, 375, or 550 can be (1) turned off to reduce the temperature to ambient conditions, (2) lowered to the maximum ambient temperature for which the detection system is specified (e.g., 30° C.) to maintain a constant storage temperature, or the temperature controller may actively cool the containers. In some embodiments, multi-well reagent containers are not commonly stored in the detection system, reducing the need for differing temperature control when the detection system is idle. Regulating the temperature during the measurement process may be advantageous to, for example, make the detection system less sensitive to variations in ambient temperature due to detection-method specific mechanisms. For example, electrochemiluminescence is temperature sensitive (e.g., see U.S. Pat. No. 5,466,416).

In an exemplary operation, holder 115 can hold container 373 as well as possibly regulating its temperature. Pipeftor 405, under the control of motion control system 102, can aspirate a sample from sample container 320 and can dispense the sample into a well of container 373. The well can contain dry-binding reagents specific for a particular analyte of interest that may be present in the sample. Following an incubation period, the incubated mixture may undergo a free-bound separation and sample matrix removal in prewash apperatus 220 before being aspirated into flow cell 192. Probe 150 may be positioned in fluid-handling manifold 425 so as to aspirate and/or dispense one or more reagents and introduce them into flow cell 192. The movement of fluids may be controlled through pump 870, and the selection of reagents aspirated from fluid-handling manifold 425 may be controlled by valves 470 and sensors 500 operating so as to send an error message to computer system 101 if a reagent line becomes empty. Optionally, pipettor 405 may also be used to combine one or more samples and/or one or more reagents in the well of container 373 (e.g., to carry out assay reactions prior to introduction of samples into flow cell 192).

Assay measurements may be conducted on samples and/or assay reaction mixtures in flow cell 192. Computer system 101 may receive data and carry out data analysis. After completion of a measurement, flow cell 192 may be cleaned and prepared for the next measurement. The cleaning process may include the introduction of cleaning reagents into flow cell 192 by directing pipettor 405 and pump 870 to aspirate cleaning reagents from fluid-handling manifold 425.

In an exemplary operation, holder 1501 can hold container 550 as well as possibly regulating its temperature. Pipettor 405, under the control of motion control system 102, can aspirate a sample from sample container 320 and dispense the sample into a well of container 550. That well can contain dry-binding reagents specific for a particular analyte of interest that may be present in the sample. Following an incubation period, the incubated mixture may undergo a free-bound separation and sample matrix removal in prewash apperatus 220 before being aspirated into flow cell 192. Probe 150 may be positioned in a reagent cavity of container 550 so as to aspirate and/or dispense one or more reagents and introduce them into flow cell 192. Air can be aspirated through probe 150 by the pipettor 450 raising probe 150 out of the reagent cavity and into air. The movement of fluids may be controlled through pump 870, and the selection of reagents aspirated may be controlled by pipettor 450. Optionally, pipettor 405 may also be used to combine one or more samples and/or one or more reagents in the well of container 550 (e.g., to carry out assay reactions prior to introduction of samples into flow cell 192). Assay measurements may be conducted on samples and/or assay reaction mixtures in flow cell 192. Computer system 101 may receive data and carry out data analysis. After completion of a measurement, flow cell 192 may be cleaned and prepared for the next measurement. The cleaning process may include the introduction of cleaning reagents into flow cell 192 by directing pipettor 405 and pump 870 to aspirate cleaning reagents from container 550.

F. Prewash

A prewash apparatus can be used for one or more of the following reasons: (1) to separate label that is not linked to magnetizable beads from the label that is linked, or (2) to remove the sample matrix from the incubated sample so that the sample matrix does not contact the measurement zone (e.g., an electrode used in a electrochemiluminescence measurement). Label separation (sometimes referred to as free-bound separation), is an important part of many assay systems in order to differentiate label that has interacted with the analyte from label that has not. Non-specific binding of labeled binding reagents in the measurement zone can be reduced with a prewash apparatus. Removal of sample matrix can also be important. For example, in electrochemiluminescence measurements, proteins and lipids from the sample matrix can absorb to the electrodes, which can change their impedance and ultimately affect the amount of measured luminescence.

In some embodiments, the sample can rehydrate dry binding reagents, where, for example, only the sample performs this rehydration. Thus, analytes in the sample are not diluted, which can reduce incubation times. On the other hand, these non-diluted samples would also have non-diluted sample matrices, the effects of which can be mitigated by the prewash apparatus.

In certain embodiments that use magnetizable beads in the binding reagents, the detection system is equipped with prewash apparatus 220. The prewash apparatus forms a first magnetic capture zone with flow cell 192 having the second magnetic capture zone. In use, after forming and incubating a composition comprising a sample of optionally processed liquid from the sample container and binding reagents comprising a plurality of magnetizable beads, a plurality of labels, and a plurality of reagents specific for an analyte of interest, the incubated composition can be aspirated from the assay well into the prewash apparatus. The beads are captured in the prewash apparatus with a magnet, and the non-captured components of the incubated composition are dispensed to a waste location. Optionally, additional liquid can be used to wash the captured beads by dispensing into the waste location. Afterwards, the captured beads can be released and moved into the measurement zone (e.g., in flow cell 192) and label that is bound to the beads can be measured. In some embodiments, the waste location is the assay well that the incubated composition originated. In some embodiments, the waste location can be decontaminated by dispensing a decontamination reagent (e.g., a sodium hypochlorite) into the waste location.

In some embodiments, the liquid from the sample container is not processed, and the sample is simply the liquid in the sample container. In other embodiments, there is a processing step on the liquid in the sample container before a sample is taken from it to help form an incubated composition. This sample pre-processing is discussed in detail, infra, and may include, filtering the liquid, centrifuging the liquid, diluting the liquid, lysing cells that may be present in the liquid, and/or releasing proteins, nucleic acids, or other analytes that may be bound to other components in the liquid.

In some embodiments, during the incubation of the incubated composition, the assay well can be agitated. This agitation can, for example, help reduce incubation times. Agitation can be accomplished using means described below, and include a simple one dimensional agitator. In some embodiments, during the incubation of the incubated composition, the assay well can be held at an elevated temperature, to provide, for example, a reduced incubation time or a more consistent reaction kinetic. In some embodiments the assay well can be simultaneously agitated and held at an elevated temperature to reduce incubation times and provide consistent reaction conditions.

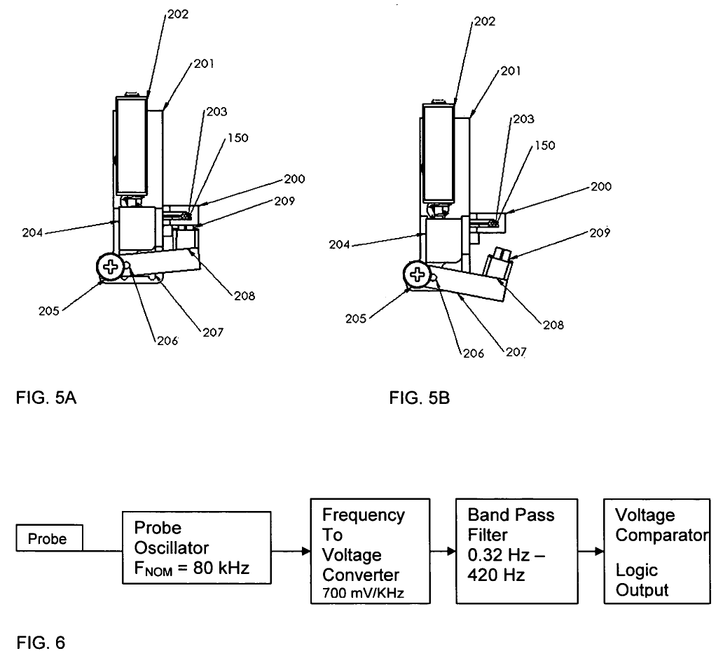

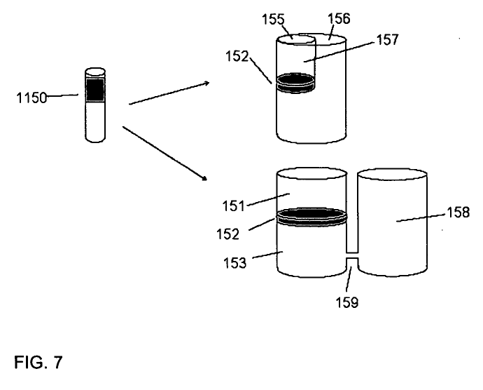

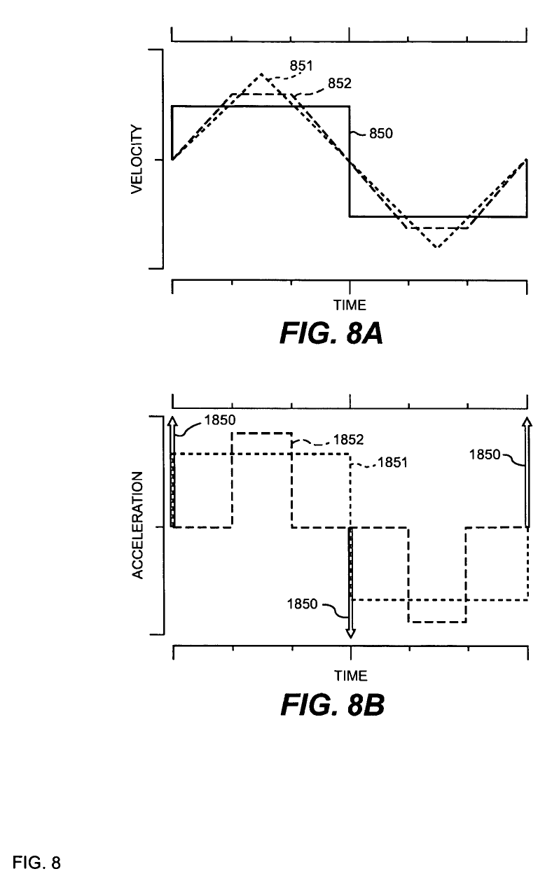

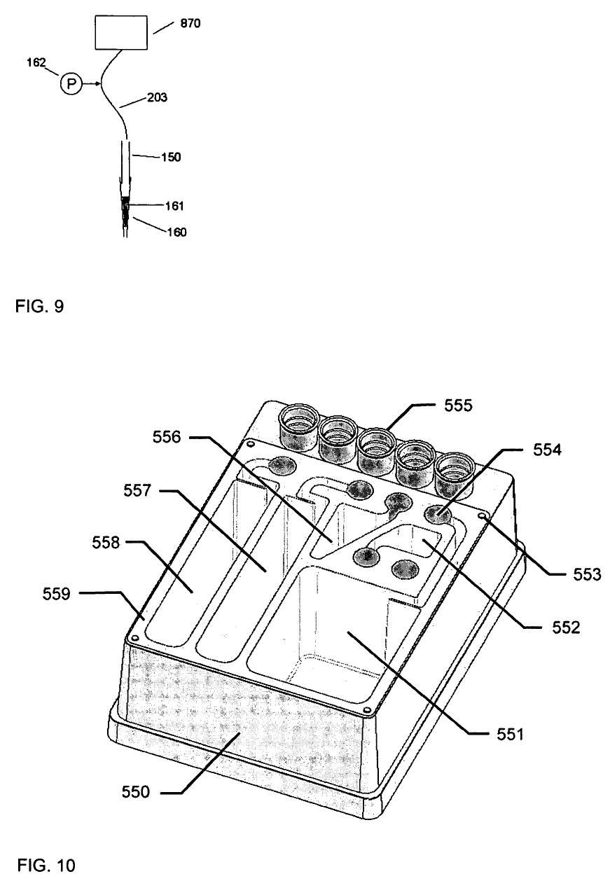

Turning now to FIGS. 4A, 4B, 5A, and 5B, probe 150 can be fluidically connected to tubing 203. Tubing 203 can go through the prewash apparatus 220, being held in place by tubing holder 200. Magnet set 209 can be moved relative to tubing 203 to exert substantial or minimal magnetic forces on magnetizable beads found in tubing 203. Motion of magnet set 209 can be performed using a solenoid 202 and a pivot arm 207. Force from solenoid 202 can be transmitted through a solenoid actuator with coupling spring 204 to pin 206 that links solenoid 202 to pivot arm 207. Magnet holder 208 can connect pivot arm 207 to magnet set 209. Plate 201 can hold solenoid 202, a shoulder screw 205, and a tubing holder 200 together. Magnet set 209 can be one or more individual magnets. Each magnet can have 2 or more magnetic field sources, for example, 3, 4, 5, 6, 7, 8, 9, 10, 11, 12, or more field sources. In some embodiments, at least one magnet can be a sandwich magnet. In some embodiments, 1, 2, 3, 4, 5, or 6 sandwich magnets can be used in magnet set 209. Some magnets suitable for use in magnet set 109 are described in U.S. Pat. No. 5,744,367. Exemplary magnetic field sources have a maximum energy product (BHₘₐₓ) of at least 3×10⁴ J/m³, for example, at least 1×10⁵ J/m³. As shown, magnet set 209 consists of 4 sandwich magnets each having two neodymium-iron-boron magnetic field sources with a BHₘₐₓ of 3.5×10⁵ J/m³ separated by a core of vanadium permendur. The distance that magnet set 209 has to be moved to substantially change the force on magnetizable beads in tubing 203 depends on the magnetic configuration. Sandwich magnets and channel magnets can reduce that distance by reducing the magnetic field strength at locations distant from the magnet, which in turn may simplify the design of the moving parts of the prewash mechanism. In some embodiments, a sensor can be incorporated to verify the location of magnet set 209 (e.g., a Hall effect sensor mounted to tubing holder 200).

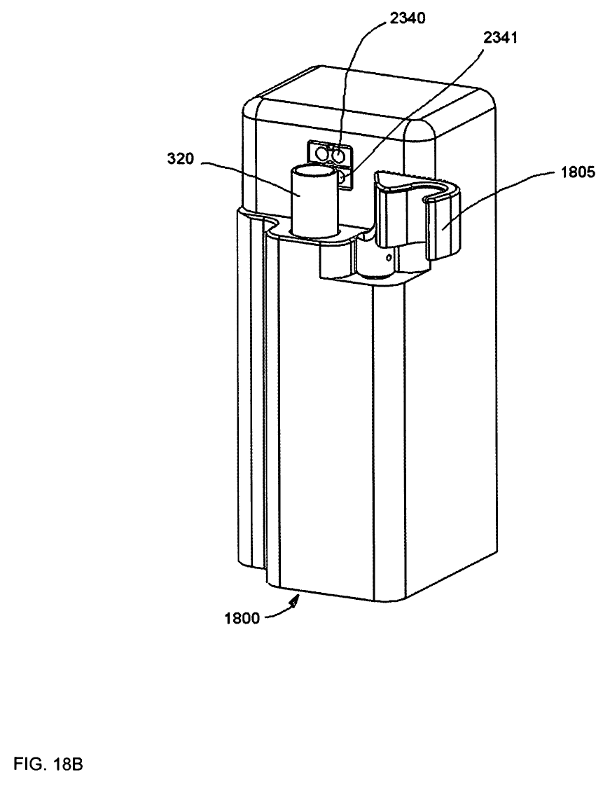

Another exemplary embodiment of a prewash apparatus is shown in FIGS. 16A and 16B. Tubing 203 can go through the prewash apparatus 220, being held in place by tubing holder 1604 and tubing enclosure 1605. A magnet set 209 can be moved relative to tubing 203 to exert substantial or minimal magnetic forces on magnetizable beads found in tubing 203. Motion of magnet set 209 can be performed using a solenoid 202 and a pivot arm 1607. Force from solenoid 202 can be transmitted through a solenoid actuator with coupling spring 204 to pin 1603 that links solenoid 202 to pivot arm 1607. Return spring 1609 helps lower magnet set 209 when minimal magnetic forces on magnetizable beads found in tubing 203 are desired. Magnet holder 208 can connect pivot arm 1607 to magnet set 209. Enclosure 1608, enclosure 1605, and enclosure 1606 can enclose solenoid 202, tubing 203, and all the moving parts of the prewash apparatus. Magnet set 209 can be one or more individual magnets, as above. As shown, magnet set 209 consists of 1 dipole magnet of neodymium-iron-boron with a BHₘₐₓ of 3.6×10⁵ J/m³. Optionally present is release magnet set 1601. Release magnet set 1601 can have the same or different composition as magnet set 209. Release magnet set 1601 can be arranged, through geometry or magnetic properties or both, (1) to negligibly affect magnet set 209's ability to collect magnetizable beads found in tubing 203 when magnet set 209 is in its close position with respect to tubing 203, and (2) to move magnetizable beads from their captured state along the wall of tubing 203 near magnet set 209 when magnet set 209 is in its far position with respect to tubing 203. Thus, release magnet set 1601 can be used to pull magnetizable beads from the tubing wall into a more central region of the cross-section of tubing 203, where fluid flow rates are faster. Thus, release magnet set 1601 can be used to improve the efficiency of removing washed beads from prewash apperatus 220 so they can be delivered to flow cell 192 and minimize carryover in prewash apparatus 220. Carryover is also minimized in prewash apperatus 220 by capturing the beads on the very smooth surface of tubing 203.

Another exemplary embodiment of a prewash apparatus uses an electromagnet rather than permanent magnets. Advantageously, electromagnets do not require moving parts.

G. Liquid Level Detection

The detection system may employ a liquid-level detector for determining the presence and/or liquid level in the sample container. A liquid level detector (LLD) may be useful for at least one of the following reasons. The LLD may help to detect the presence of a sample in the sample container. If an empty sample container is presented to the detection system, the LLD will fail to find liquid and thereby be able to warn the operator and prevent an erroneous result. The LLD can similarly be used to determine if sufficient sample is present in the sample container. The LLD may help to minimize the contact of the outside of a probe with the sample, which can reduce the amount of carryover among samples. The LLD may help to aspirate from a pre-determined distance below the top of the sample. The amount of sample and therefore the height of sample in the sample container may not be known to the detection system; therefore the location of the top of the sample would have to be measured with a LLD if that height needs to be known. Some samples are not homogenous. For example, in a centrifuged tube of whole blood, plasma occupies the top portion of the sample, and packed red blood cells occupy the bottom portion. If the desired sample is plasma, then probe 150 should aspirate near the top of the sample. The LLD may help to verify that the sample was aspirated by comparing the liquid level before and after aspiration. Capillary forces can add hysteresis to the probe position for entering and leaving the liquid sample (see, e.g., Physical Chemistry of Surfaces, 6th edition, Adamson & Gast, John Wiley & Sons, Inc. (1997)). This hysteresis can be measured and compensated for, or the probe can measure the liquid level from the same direction both before and after sample aspiration: as the probe is lowered into the sample measure the pre-aspiration sample height, aspirate the sample, raise the probe out of the sample, lower the probe again into the sample to measure the post-aspiration sample height. By knowing the cross-sectional area of the sample container, these heights can be used to compute the aspirated volume.

In some embodiments, LLD is only used with the sample in the sample container (e.g., a Vacutainer® after its cap has been removed, or any open container). In some embodiments, LLD is used for all aspirations from the sample container and the multi-well reagent containers. In some embodiments, seals are removed or a sufficiently large hole is made in the seal (e.g., through multiple piercing by a probe) that the seal does not contact the probe during LLD. In some embodiments, the probe can contact a seal and LLD can be robust to this contact.

The LLD may use an optical arrangement to interrogate the liquid height in the container.

The detector may use a probe 150 to measure the liquid level by measuring a physical change occurring at the probe tip upon contact with a liquid surface. In some embodiments, the detection system comprises means for applying an electrical signal on the probe and means for measuring a change in the electrical signal. For example, the measured change in the electrical signal can result from a measurement of at least one of (i) a DC potential, (ii) an AC potential, (iii) a DC current, (iv) an AC current, (v) a DC charge, (vi) an AC charge, and (vii) a frequency.

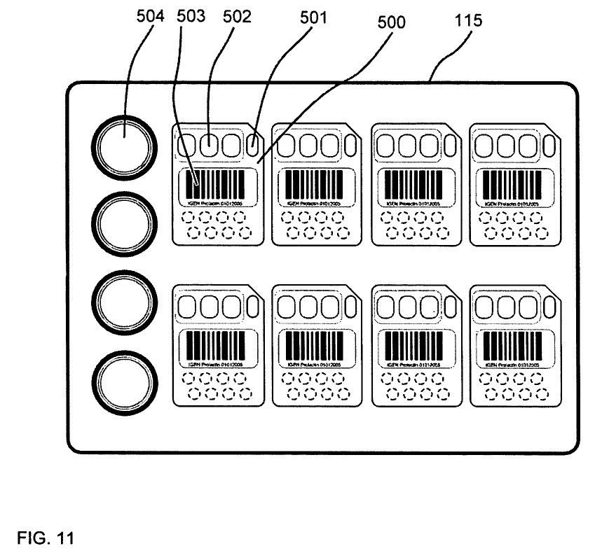

In some embodiments, an increase in capacitance resulting from liquid contact may be used. For example, the QProx™ QT301 or QT117L (Quantum Research Group, PN) can measure a change in charge associated with the additional capacitance that results when the probe contacts liquid. For example, the AD7745 (Analog Devices, (Norwood, Mass.)) is a 24 bit capacitance-to-digital converter that has a resolution of 4 aF and an accuracy of 4 fF. Thus, very small changes in the capacitance of the probe can be measured with a digital interface. In some embodiments, probe 150 may be made part of an oscillator circuit whose frequency depends on the capacitance of probe 150. When probe 150 contacts the liquid, a frequency shift occurs due to the increased capacitance. A block diagram for this approach is shown in FIG. 6. The liquid level detection circuit may be divided into 4 functional blocks: probe oscillator, frequency to voltage converter (FVC), band pass filter, and a voltage comparator/logic output that may generate an LLD signal. The probe oscillator can be a bistable oscillator constructed, for example, on an LM6132 (National Semiconductor, CA) op-amp. Hysteresis from positive feedback can set an upper and lower voltage boundary; the negative feedback path can drive the probe capacitance and set the slope of the signal that appears on the negative input to the op-amp. A second op-amp sets a low impedance reference point for the oscillator that is at 50% of the 5 V power supply (required for single supply operation). The frequency to voltage converter may convert the output frequency of the probe oscillator to a voltage. The FVC can be built on an LM331 (National Semiconductor, Calif.), with components selected to have a gain of about 56 V per 80 kHz at a nominal frequency of 80 kHz. A band pass filter can follow the FVC, reducing the FVC output to a voltage near half the supply voltage, while maintaining the high sensitivity to capacitance change and the ability to reject slow changes in capacitance as the probe moves in the detection system. The low pass nature of the band pass filter can attenuate high frequency noise and the oscillator's fundamental frequency. The effects of slow capacitance changes, which are caused by the changing position of the probe, can be filtered out by the high pass characteristic of the BPF. Quick changes in capacitance can be transmitted through the band pass filter, causing voltage changes that trigger the comparator circuit to signal either entering or leaving the liquid sample.

In some embodiments, the probe can comprise two conductors insulated from one another until the liquid sample closes the circuit. In this case, a DC or AC voltage may be applied between the conductors and a change in DC or AC current measured. These embodiments offer some robustness to sealed containers.

In some embodiments, the detection system comprises means for applying a mechanical signal on the probe and means for measuring a change in the mechanical signal. For example, the measured change can result from a measurement of at least one of (i) an amplitude and (ii) a frequency.

In some embodiments, liquid level detection can be accomplished by mechanically driving the probe at ultrasonic frequencies. The amplitude of motion is modulated by the differing mechanical impedances of a liquid sample and air. Accordingly, a marked change in amplitude of motion of probe 150 can be detected when probe 150 encounters the higher mechanical impedance of a liquid sample.

H. Agitation

In some embodiments consistent with the principles disclosed herein, the multi-well reagent containers can be agitated. Agitation may accelerate the rate of the binding reaction by stimulated convective fluid transport in the container. In some embodiments, the binding reagents comprise components that separate due to density differences. For example, at least a portion of the 2.8 μm magnetizable beads (DYNAL M-280; Invitrogen, Calif., USA) may have a density of about 1.4 g/cm³ and settle at a rate of about 1 μm/s in water. Agitation can keep the various components well mixed to accelerate the rate of the binding reactions. In some embodiments, the multi-well reagent container holder can be agitated while the sample container holder is not. This may be done, for example, to reduce the mass that must be agitated or to ease mechanical packaging. Further reducing the mass to be agitated, in some embodiments, only the assay wells in the multi-well reagent containers are agitated. In some embodiments, both the multi-well reagent container holder and sample container holders can be agitated; this may be done, for example, because the two holders are the same, or to ease mechanical packaging.

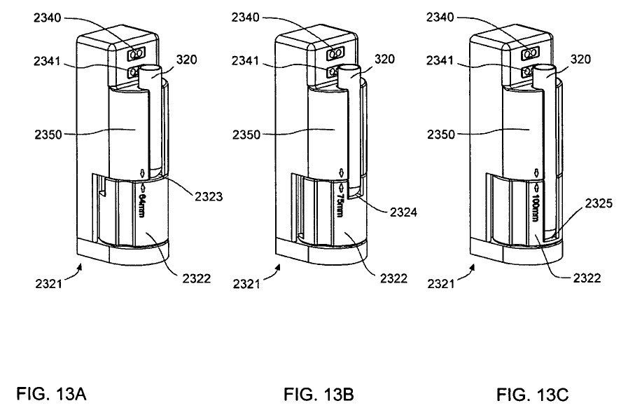

In some embodiments, the agitator of the multi-well reagent containers can move in substantially one dimension. FIGS. 8A and 8B show three example profiles that can be used to control linear reciprocation of tray 110. FIGS. 8A and 8B show the velocity (FIG. 8A) and acceleration (FIG. 8B) for one period of a profile comprising a single fundamental frequency, where both boundary points of the period are shown for clarity (if a function has a period T, then time axis t for one period would be tₒ≦t<tₒ+T for any tₒ; for clarity the time axis has been extended to tₒ≦t<tₒ+T). Profiles with multiple fundamental frequencies may also be possible, where multiple fundamental frequencies can be separated in time (e.g., a first set of single or multiple fundamental frequencies followed by a second set of different single or multiple fundamental frequencies, etc., the number of sets being greater than 1) or superposed at the same time by adding the individual time waveforms together. A velocity profile 850 may have a corresponding acceleration profile 1850. The large amplitude, short duration accelerations that accompany a step change in velocity may be represented by impulses. Similarly, velocity profile 851 may have a corresponding acceleration profile 1851 and velocity profile 852 may have a corresponding acceleration profile 1852. The acceleration profiles are related to their respective velocity profiles by mathematical differentiation.

The three profiles shown in FIGS. 8A and 8B are all piecewise constant in either velocity or acceleration. Velocity profile 850 can be piecewise constant with two piecewise constants having one positive and one negative value. While velocity profile 851 of FIG. 8A is not piecewise constant, the associated acceleration profile 1851 is piecewise constant with the two piecewise constants having one positive value and one negative value. Acceleration profile 1852 may be piecewise constant with three piecewise constants having one positive value, one negative value and one zero value. One skilled in the art can readily ascertain that many piecewise constant profiles can be generated, varying in the magnitude, number, and location of the piecewise constants as well as varying with respect to the time for one period. For example, velocity profiles 850, 851, and 852 may be modified to have a constant zero velocity component at each point where the velocity crosses zero (i.e., when the reciprocation is changing directions). If drive mechanism 178 is a stepping motor, then small changes in the continuous-time velocity and acceleration profiles shown in FIGS. 8A and 8B may occur due to the quantized step rate of motor 178.

In certain embodiments, controller 101 may be configured to control linear reciprocation of the tray to have either a piecewise constant velocity profile or a piecewise constant acceleration profile in which the number of piecewise constants does not exceed 24. According to another embodiment, the number of piecewise constants may not exceed 12. In further embodiments, the number of piecewise constants may equal two or three. It should be appreciated that the computational complexity of generating the appropriate timing to drive a motor may be smaller when only the velocity and acceleration are controlled for a given displacement. This general-purpose motion control may need only minimal adaptation between moving at least a multi-well reagent container from an extended position to inside the biological detection system and moving the container in an approximately sinusoidal manner. Furthermore, the amount of harmonic content in the agitation may be modified by selecting a velocity and/or acceleration that closely or more distantly approximates a sinusoid. During agitation, it may be desirable to minimize the accelerations that the rest of the detection system experiences during agitation and prevent the samples from splashing out of the container, while ensuring that the agitation achieves satisfactory mixing of the samples.

According to some embodiments disclosed herein, the controller 101 may be configured to control linear reciprocation of the tray using a profile that is trapezoidal in shape, similar to velocity profile 852. According to some embodiments, each wavelength of a trapezoidal profile can include increasing positive velocity component, a constant positive velocity component, a decreasing positive velocity component, a decreasing negative velocity component, a constant negative velocity component, and an increasing negative velocity component. According to certain embodiments, each of these six components can be approximately equal in duration. According to one embodiment, the linear reciprocation can have a fundamental frequency of approximately 20 Hz, an amplitude of approximately 3 mm, and a 5^{th} harmonic being second only to the fundamental frequency in amplitude.

In some embodiments, the agitator of the at least one multi-well reagent container can move in two dimensions. For example, the agitator may have a substantially circular motion or substantially elliptical motion. In further embodiments, the agitator may move at least one multi-well reagent container in a more complex orbit.

In some embodiments, the agitator can be an eccentric mass on a DC motor, mechanically coupled to at least the assay wells of the multi-well reagent containers.

I. Multi-Well Reagent Containers

Multi-well reagent containers contemplated herein may be described by both their structure and their content. Structurally, the wells of a multi-well reagent container can be formed from one part or multiple parts.

1. Multiple Parts

When formed from multiple parts, the parts can be one or more vessels and a receptacle, wherein the receptacle is adapted to receive each of the vessels and further comprises zero or more reagent cavities. When speaking collectively, the vessels and reagent cavities are termed “wells”. The vessels can be held in the receptacle via an attachment retention member, so that, for example, the vessels remain in place under accelerations as large as 10 times that of gravity. The attachment retention member can be an ultrasonic weld, a snap fit (such as a permanent snap fit), or other techniques as known in the art. The attachment retention member can be configured to hold the vessels rigidly in the receptacle or to hold the vessels loosely so that the vessel bottom can move a greater distance that the vessel opening (e.g., by tilting), which may be useful for agitating only the assay wells.

2. One Part

In embodiments where the wells of the multi-well reagent container are formed from one part, at least one of the wells can be an assay well and at least zero of the wells can be non-assay wells, with the total number of wells being at least 2. In some embodiments where the wells of the multi-well reagent container are formed from multiple parts (hereafter, multi-part multi-well reagent containers), all assay wells are vessels. In some embodiments using muti-part multi-well reagent containers, all wells are vessels (i.e., there are no reagent cavities). In some embodiments using multi-part multi-well reagent containers, all non-assay wells are reagent cavities. In some embodiments using multi-part multi-well reagent containers, at least one assay well is a vessel. In some embodiments using multi-part multi-well reagent containers, at least one non-assay well is a reagent cavity.

3. Content

By content, the wells of a multi-well reagent container can be assay wells or other wells (collectively termed non-assay wells). Non-assay wells can be used for a variety of purposes. For example, a non-assay well may be empty, so as to operate as a sample container that for, example, the operator can pipette the sample into. Empty wells can also be used by the detection system as a staging location for a multi-step assay. Non-assay wells can also be used to hold a positive control/calibrator or a negative control/calibrator that can be (after rehydration if dry) pipetted into an assay well to form an assay control/calibrator. Altematively, an assay well can further comprise an assay control/calibrator. Non-assay wells can also be used to hold liquid reagents that (a) are not specific to the analyte of interest and (b) assist in the detection of the label. For example, non-assay wells can hold an ECL coreactant containing liquid, e.g., BV-GLO™ Plus (BioVeris Corporation, Gaithersburg, Md.). Non-assay wells may hold a cleaning solution for flow cell 192, e.g., BV-CLEAN™ Plus (BioVeris Corporation, Gaithersburg, Md.). Non-assay wells may hold a rehydrating solution for a dry composition (e.g., a positive control/calibrator, a negative control/calibrator, or binding reagents). Non-assay wells may hold decontamination reagents (e.g., used to decontaminate a used multi-well reagent container or the detection system) such as bleach (e.g., a hypochlorite solution, or hypochlorite in a basic solution). Non-assay wells may hold reagents used to assist in the binding reactions by making the analyte accessible: for example, (a) lysing agents such as diethylene glycol, hydrogen peroxide, saponins, surfactants, (b) releasing agents such as acetonitrile used for example to release 25-hydroxy vitamin D from binding proteins, or (c) extraction buffers to reduce non-specific binding of the analyte such as a solution having a pH ≧8 or a pH≦6 and a osmolarity greater than or equal to 0.1 osmol/L or a solution having an osmolarity greater than 1.1 osmol/L (see U.S. patent application Ser. No.11/303,999). Other extraction buffers include those useful for extracting antigens from larger entities, such as nitrous acid or precursors of nitrous acid in 2 non-assay wells such as an acid (e.g., acetic acid) in one non-assay well and a dry nitrate salt in the other non-assay well. Nitrous acid can be used to extract cell wall antigens from gram positive bacteria and may also be useful in extracting antigens from other organisms in mucus-containing samples such as upper respiratory samples. Another exemplary extraction buffer comprises a non-ionic alkyl-polyoxyethylene detergent of general formula R—(OCH₂CH₂)ₙ—O-Z, where (i) R is —H or —CH₂; (ii) n is an integer greater than 2; and (iii) Z is an alkyl group, for example, —(CH₂)ₘCH₃, where m is between 7 and 17, for example H—(OCH₂CH₂)₁₂—O—(CH₂)₁₁CH₃ also known as Laureth-12. These detergents can be useful for example, for exacting antigens from cryptosporidium oocytes (e.g., C. parvum oocytes). Non-assay wells may hold analyte-protecting reagents such as protease inhibitors, non-specific DNA or other nucleic acid containing compounds (e.g., to minimize effects of endogenous nucleases on nucleic acid tests), or nuclease inhibitors. Non-assay wells may hold non-analyte specific label such as e.g., labeled anti-human IgG. Other reagents that non-assay wells may hold include fixative agents, reducing agents, oxidizing compounds, pH modifiers (such as Schiffs base, organic and inorganic acids and bases), delipidating compounds (such as lipases and 1,1,2 trichlorofluoroethane), proteolytic enzymes or proteases, nucleases, blocking agents, aspartame or other rheumatoid factor inhibiting compounds, and clotting activators (such as calcium to enable rapid measurements of activated partial thromboplastin time or APTT).

In some embodiments, the multi-well reagent container comprises dry reagents and liquid reagents. Typically, the dry reagents started as liquid reagents and were subsequently freeze-dried. The manufacturing yield on the freeze-drying can be less than 100%. Having a multi-part multi-well reagent container where at least one of the vessels comprises dry reagents may improve the overall yield of the container by testing lots of vessels before assembling into the container. Assuming the number of vessels with differing lots of dry reagents is n and the probability of failure is p and np is small, the amount of failed material is p versus np for the individual lot testing compared to testing assembled multi-well reagent containers. Thus, in some embodiments, all dry reagents can be in vessels. Because liquid reagents lack the freeze-drying step, the probability of failure in filling reagent cavities can be sufficiently small that the cost of assembling a multi-well reagent container from additional vessels is larger than the cost of failing filled receptacles. Thus, in some embodiments, all liquid reagents can be in reagent cavities. In some embodiments, dry reagents can be in vessels and liquid reagents can be in reagent cavities.

4. Seals

In some embodiments, the multi-well reagent containers can be hermetically sealed. In some embodiments, each vessel is individually hermetically sealed. In some embodiments, the reagent cavities can be hermetically sealed. In some embodiments, the container may be sealed with an elastomeric, thermoset, or a thermoplastic material, such as EVA or Santoprene®, that has been pressed into the container's openings. In some embodiments, the container may be sealed with a laminate comprising a metallic layer, such as a foil microplate seal. In various embodiments, the container may be sealed with a laminate comprising a thermally modifiable layer, such as a laminate that can be heat-sealed to the container. In some embodiments, the container may be sealed with a laminate comprising an adhesive layer that can bond the laminate to the container.

5. Enclosures

In some embodiments, the multi-well reagent container can comprise at least one enclosure, such as one or more sealed enclosures (containers) inside a sealed bag. In some embodiments, the sealed bag may be comprised of, for example, polyethylene, polyester, aluminum, nickel, a trilaminate of polyester-foil-polyethylene, or a bilaminate of polyester-polyethylene. In some embodiments, a desiccant may be added between the innermost enclosure and the outermost enclosure. The desiccant may, for example, comprise calcium oxide, calcium chloride, calcium sulfate, silica, amorphous silicate, aluminosilicates, clay, activated alumina, zeolite, or molecular sieves.

In some embodiments, a humidity indicator may be added between the innermost enclosure and the outermost enclosure. The humidity indicator may, for example, be used as an indication that the dry composition remains sufficiently dry such that its stability has not been compromised. In some embodiments, the humidity indicator may be viewed through the outermost enclosure. In certain embodiments, the humidity indicator may be a card or disc wherein the humidity is indicated by a color change, such as one designed to meet the U.S. military standard MS20003. In some embodiments, the humidity barrier created by the container can be sufficient to keep a dry composition in a well dry when the temperatures are 45° C., 25° C., or 4° C. and the conditions are 100% relative humidity for 10 days, 20 days, 40 days, 67 days, 3 months, 6 months, 12 months, 18 months, 24 months, or longer.

6. Assays

In some embodiments, each assay well in a multi-well reagent container can hold binding reagents specific for only one analyte of interest, and each assay well can hold binding reagents specific for the same analyte of interest. In some embodiments, each assay well can hold identical reagents. In some embodiments, each assay well can hold binding reagents specific for the same analyte of interest, with some assay wells additionally comprising positive and/or negative control/calibrator materials. In some embodiments, each assay well can hold binding reagents specific for the same analyte of interest, with some non-assay wells comprising positive control/calibrators and/or negative control/calibrators. In some embodiments, the container may only be partially consumed by each test; consequently, the container may not have to be replaced after every sample—leading to greater operator convenience.

In one embodiment, the multi-well reagent containers hold at least one control/calibrator well and at least one assay well for at least one analyte of interest. In some embodiments, the container can comprise two control/calibrator wells for a two-point calibration, and seven identical assay wells for seven samples and/or duplicated measurements. In some embodiments, the multi-well reagent containers may hold 1, 2, 3, 4, 5, 6, 7, 8, 9, 10, 11, 12, 13, 14, 15, or more control/calibrator wells. In some embodiments, the multi-well reagent containers may hold 7 or more identical assay wells for sample measurements. In some embodiments, the multi-well reagent containers may hold 1, 2, 3, 4, 5, 6, 8, 9, 10, 11, 12, 13, 14, 15, 16, 17, 20, 23, 25, 34, 36, 47, 49, 62, 64, 79, 81, 94, 96, 382, 384, or more identical assay wells for sample measurements.

In some embodiments, each assay well in a multi-well reagent container can hold binding reagents specific for only one analyte of interest, and the container can hold binding reagents specific for at least two different analytes of interest. Consequently, these containers may be used to test for multiple analytes of interest. In some embodiments, each assay well can hold binding reagents specific for different analytes of interest. In some embodiments, multiple assay wells can hold binding reagents specific for the same analyte of interest; the container can have, for example, one or more control/calibrator assay wells and one or more sample measurement assay wells. In some embodiments, multiple assay wells can hold binding reagents specific for the same analyte of interest; the container can have, for example, one or more control/calibrator non-assay wells and one or more sample measurement assay wells. In some embodiments, the container may only be partially consumed by each test; consequently, the container may not have to be replaced after every sample—leading to greater operator convenience.

In some embodiments, the container can comprise two control/calibrator wells for a two-point calibration for each of three analytes of interest, and one sample assay well for each of the same three analytes of interest. In some embodiments, the multi-well reagent containers may hold two-point calibration wells for 1, 2, 3, 4, 5, 6, 7, 8, 9, 10, 11, 12, 13, 14, 15, or more analytes of interest. In some embodiments, the multi-well reagent containers may hold one-point calibration assay wells for 1, 2, 3, 4, 5, 6, 7, 8, 9, 10, 11, 12, 13, 14, 15, or more analytes of interest. In some embodiments, the multi-well reagent containers may hold three-, four-, or five-point calibration wells for 1, 2, 3, 4, 5, 6, 7, 8, 9, 10, 11, 12, 13, 14, 15, or more analytes of interest. In some embodiments, the multi-well reagent containers may have two wells that serve as assay control/calibrators for three analytes of interest. In some embodiments, the multi-well reagent containers may have a set of assay wells (e.g., 1, 2, 3, 4, 5, 6, 7, 8, 9, or 10) that serve as assay control/calibrators for multiple (e.g., 1, 2, 3, 4, 5, 6, 8, 9, 10, 11, 12, 13, 14, 15, 16, or more) analytes of interest. These embodiments that share assay control/calibrators across analytes of interest may take advantage of situations wherein the greatest variability in signal levels from measurement of the label result from non-analyte specific mechanisms (e.g., storage environment of the container, non-analyte specific interference in the sample).

In some embodiments, the multi-well reagent containers may have sample assay wells specific for 1, 2, 3, 4, 5, 6, 7, 8, 9, 10, 11, 12, 13, 14, 15, 20, 23, 25, 34, 36, 47, 49, 62, 64, 79, 81, 94, 96, 382, or 384 analytes of interest. In some embodiments, the multi-well reagent containers may have sample assay wells specific for sixteen or more analytes of interest. In some embodiments, the container can comprise two control/calibrator wells for a two-point calibration that is shared across seven analytes of interest, and one sample assay well for each of the same seven analytes of interest. In some embodiments, the container can comprise two control/calibrator wells for a two-point calibration that is shared across 1, 2, 3, 4, 5, 6, 7, 8, 9, 10, or more analytes of interest, and one sample assay well for each of the same analytes of interest. In some embodiments, the container can comprise one control/calibrator well that is shared across four analytes of interest, and one sample assay well and one individualized control/calibrator assay well for each of the same four analytes of interest.

In some embodiments, at least one assay well in a multi-well reagent container can hold binding reagents specific for at least one analyte of interest and a control/calibrator for that at least one analyte of interest. For example, at least one assay well can contain reagents for two control/calibrators for a two-point calibration for an analyte of interest as well as reagents for a sample measurement of that analyte. In some embodiments, at least one well can contain reagents for 1, 2, 3, 4, or 5 control/calibrators for an analyte of interest as well as reagents for a sample measurement of that analyte.

In some embodiments, at least one assay well in a multi-well reagent container can hold binding reagents specific for more than one analyte of interest. In some embodiments, each assay well can comprise identical reagents. For example, each assay well may contain all the analyte-specific binding reagents and control/calibrators to measure 2, 3, 4, 5, 6, 7, 8, 9, 10, 11, 12, 13, 14, 15, 16, or more analytes of interest; perhaps requiring only one assay well per sample. In some embodiments, each assay well may contain all the analyte-specific binding reagents to measure 2, 3, 4, 5, 6, 7, 8, 9, 10, 11, 12, 13, 14, 15, 16, or more analytes of interest. In some embodiments, each assay well may contain control/calibrators to measure 2, 3, 4, 5, 6, 7, 8, 9, 10, 11, 12, 13, 14, 15, 16, or more analytes of interest. In these cases, the signal generated by the measurement of each analyte in a single assay well may be combined or may be separated. For example, if the presence of any of a plurality of analytes is desired to be detected, the signals from each analyte may be combined and jointly detected. For example, if quantitative or qualitative determination of each of the plurality of analytes is required, then the signals from each analyte may be required to be separated. This separation can occur through, for example, (1) spatial separation by linking capture reagents specific for each analyte in differing areas on the assay wells (e.g., each area being a separate electrode, perhaps comprising carbon as in U.S. patent application Publication No. 2005/0052646) and/or (2) label separation by having labels that differ in a measurable property for each analyte (e.g., color in fluorescent labels).

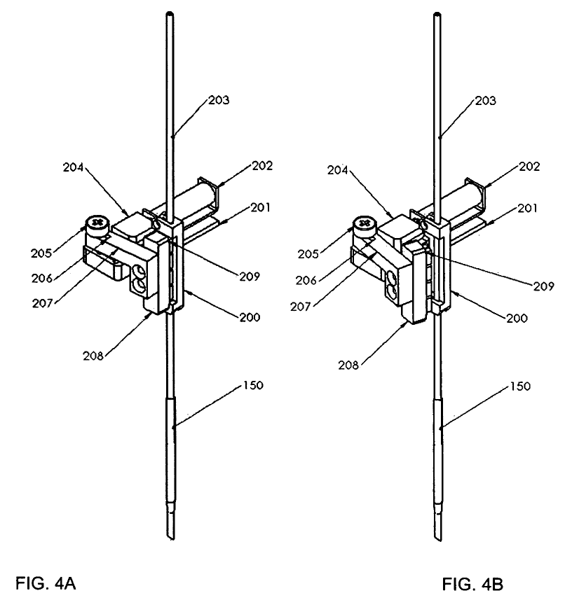

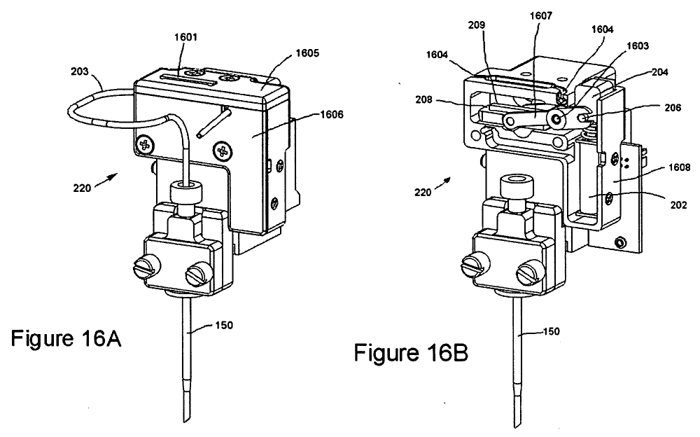

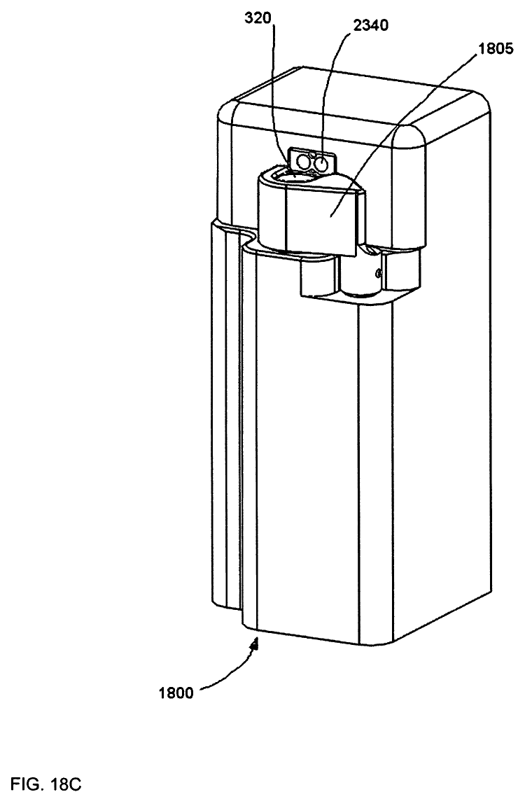

Examples of multi-well reagent containers are shown in FIG. 2, FIG. 3, FIG. 10, FIG. 11, and FIG. 17. FIG. 3 depicts one example of a 9-well multi-well reagent container, where structurally the wells of the multi-well reagent container are formed from one part. Container 373 may comprise wells 378, a sealing surface 381, an identification label 379, and finger grips 380. Seals for sealing surface 381 are described above. FIG. 2 depicts container 373 with a seal, and FIG. 2 also depicts one example of a 6-well multi-well reagent container, where structurally the wells of the multi-well reagent container are formed from one part. Container 375 is structurally analogous to container 373. FIG. 2 also shows both containers mounted in holder 115. Container 373 or container 375 can match holder 115 to the extent that each well in the container can fit into depression 117 or analogous low area. On the other hand, not every depression 117 needs to have a corresponding well for container 373 or container 375 to fit in holder 115. Matching depressions in holder 115 and with assay wells in the container can improve heat transfer in those embodiments providing temperature control of the assay wells. In some embodiments, the bottom of multi-well reagent container 373 or container 375 may be flat, and multi-well reagent container holder 115 can secure the container along its edges.

FIG. 10 depicts an embodiment for a multi-well reagent container, where structurally the wells of the multi-well reagent container are formed from multiple parts. Container 550 can comprise vessels that may be assay wells or non-assay wells. For example, three of vessels 555 can be assay wells, one can be the sample container after sample is pipetted into it by the operator, and one can be empty so as to be able to dilute the sample. As another example, all five vessels can be assay wells. As another example, the vessels can be assay wells and control/calibrators. Liquid reagents may be held in reagent cavities 552, 556, 557, and 558, for example. Examples of liquid reagents that may be used include bleach, water, BV-STORE™ (BioVeris Corp, MD), BV-DILUENT™ (BioVeris Corp, MD), BV-GLO™ Plus (BioVeris Corp, MD), and BV-CLEAN™ Plus (BioVeris Corp, MD). Waste may be held, for example, in reagent cavity 551. Reagent cavity 551 may have an absorbent material, a solidification agent, and/or a decontamination agent to ease handling and disposal of used multi-well reagent containers. To prevent spillage of the liquid reagents and waste, self-sealing probe ports 554 may be used to reversibly connect and seal the reagent cavities to probe 150. Seal 559 can prevent liquid or gas exchange from the reagent cavities, excepting through ports 554. Ports 554 may also allow air exchange to replace aspirated dispensed liquids. In some embodiments, the liquid reagents can comprise the label and/or a binding partner such as an antibody. The label and/or binding partner may not be specific for a particular analyte of interest. For example, the binding partner may be a labeled anti-human antibody while the analyte of interest may be a particular human antibody. In some embodiments, the liquid reagents may comprise dilution reagents, pretreatment reagents, releasing agents, and/or lysing agents. FIG. 15 shows two of container 550 loaded in a detection system on holder 1501. Vessels 555 can be exposed on the bottom of container 550 so that, for example, thermal contact can be directly made and/or an agitator can directly contact the vessels.

FIG. 11 depicts one example of a multi-well reagent container, where structurally the wells of the multi-well reagent container are formed from one part. Container 500 can comprise three wells 502 of liquid reagents under a seal. Well 501 may be empty and may be used to mix the reagents and sample. Identification label 503 can be used to automate the recognition of the container by the detection system. For example, the three wells 502 may contain a labeled antibody, a capture antibody, and magnetizable beads that can bind to the capture antibody, respectively. Many assay construction formats are possible with this arrangement, as demonstrated by the Elecsys® 2010 (Roche Diagnostics). In some embodiments, the multi-well reagent container holder may also be the sample container holder. Four sample containers 504 may be placed along side eight multi-well reagent containers. As another example the three sealed wells 502 may each contain the assay reagents for three distinct analytes. Sample may be pipetted into well 501 and distributed to the three wells by the analyzer for incubation and subsequent analysis.

FIGS. 17A, 17B, 17C, and 17D depict one example of a multi-well reagent container, where structurally the wells of the multi-well reagent container are formed from multiple parts. Container 1701 comprises vessels 1702 that may be assay wells or non-assay wells. For example, three of vessels 1702 can be assay wells and one can be the sample container after sample is pipetted into it by the operator. As another example, all four vessels can be assay wells. As another example, the vessels can be assay wells and control/calibrators.

Vessels 1702 comprise an opening 1704 that is sealed by seal 1705 along flange 1706. Attachment retention members 1703 form a permanent snap fit into receptacle 1711. Gap 1707 enables the vessels to move in receptacle 1711. Because the snap fit is located closer to opening 1704 than to bottom 1710, the vessel bottom can move a greater distance than the vessel opening, which may be useful, for example for agitation. Identification label 1708 can be used to automate the recognition of the container by the detection system. Seal 1709 can seal reagent cavities that are not shown, but can be similar to those in FIG. 10.

J. Sample Entry

1. Single Sample, Multi-size Holder

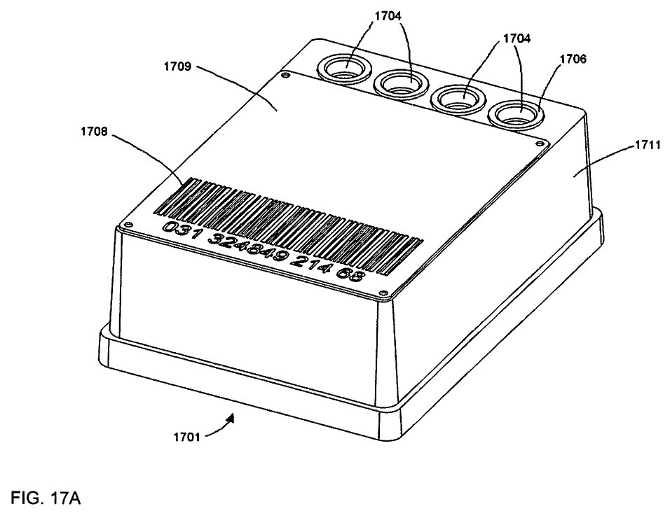

In some embodiments, the holder of a sample container 321 takes the form of holder 1321, as shown in FIG. 12. Holder 1321 can comprise four slots 1322, 1324, 1326, and 1328, for holding differing sized sample containers 320. The sample containers rest on the bottom surfaces of the slots. For example, a sample container in slot 1324 can rest on bottom surface 1325 while a sample container in slot 1322 can rest on bottom surface 1323. The bottom surfaces may be arranged so that the top of sample containers placed in the appropriate slots may be nearly the same distance from top surface 1340 (i.e., the tops of the sample containers are at a constant elevation). Having the same elevation for the tops of the sample containers can reduce the travel required for pipettor 405 to aspirate sample without limiting the detection system to a particular type of sample container. Examplary sample containers include the common 75 mm and 100 mm long Vacutainer® tubes, as well as the less-common 64 and 125 mm long tubes.

Different diameters may also be accommodated by different slots, such as 10.25, 13, and 15 mm diameter tubes. Fisher® cups of various sizes could also be accommodated. While holder 1321 is depicted with 4 slots, alternate embodiments could have more or fewer slots; further, the detection system may accept many holders of varying slots.

The slots in the holder may or may not expose a portion of the side of the sample containers. As shown in FIG. 12, the sides of the sample containers may be exposed. This exposure may be used to read a barcode label affixed to the sample container. To reduce detection system complexity, instructions (e.g., 1334 and 1332) may be included on the sides of the container to assist the operator in selecting the appropriate slot, to orient the barcode label appropriately, and to load the holder into the detection system in the correct orientation.

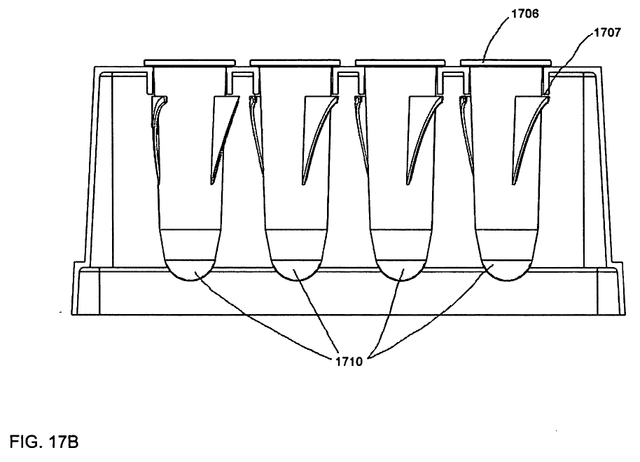

In some embodiments, a holder of a sample container 321 can take the form of holder 2321, as shown in FIG. 13. Many embodiments of holder 1321 can be removably placed in the detection system, whereas holder 2321 may be fixed in the detection system. Rotator 2322 can be equipped with detents to align the appropriate bottom surface (e.g., 2323, 2324, and 2325) with the slot in mount 2350. Together, rotator 2322 and mount 2350 can form slots similar to those of holder 1321. Sample containers 320 of varying length can be accommodated by rotator 2322. Sample container elevation may be confirmed by sensors 2341 (not too low) and 2340 (not too high). These sensors may be in the detection system for holder 1321.

Different diameters of sample container 320 may be accommodated by springs located in the slot. In some embodiments, (e.g., those that accommodate only 13 and 15 mm diameter tubes) the sample containers can be biased against one wall while the probe can be positioned to sample from the center of the largest container. The probe can still aspirate from the location of the center of the largest container as long as the probe is within the boundaries of the smallest container. In some embodiments, the slot in mount 2350 can be large enough to accommodate the largest container while different slots in rotator 2322 are sized to closely match different diameters. In this case, the bottom surfaces can be reached by selecting the correct diameter on the rotator. The diametrical information could be read from sensors (e.g., magnets) embedded in rotator 2322 in order for the detection system to recognize the sample container diameter. This information may be useful, for example, in keeping a constant amount of the probe tip submerged in the sample container while aspirating sample.

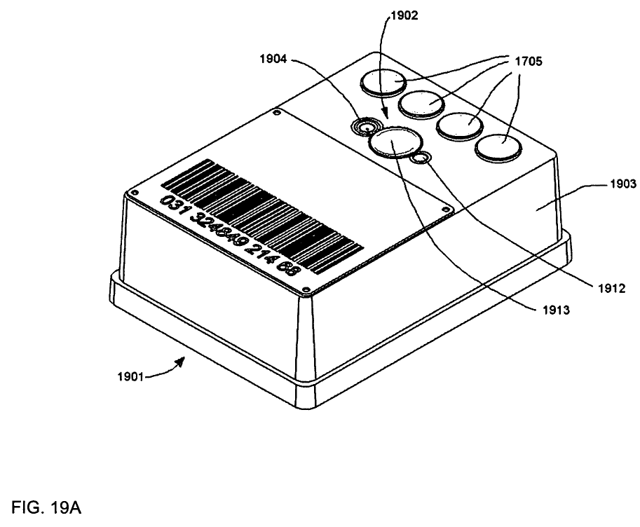

FIGS. 18A, 18B, and 18C depict another exemplary holder of a sample container 321 and holder 1800. Holder 1800 may be fixed in detection system. Holder 1800 is designed to bring any sample container between a maximum and minimum height to the same top surface elevation. Container 320 rests on platform 1801. Platform 1801 is moveable by motor 1802 via belt 1803. Electronics located on printed circuit board 1804 (or elsewhere) control motor 1802 so that platform 1801 is raised if both sensors 2340 and 2341 do not indicate the presence of a container and if the platform is not at its highest extent. If both sensors 2340 and 2341 indicate the presence of a container and if platform 1801 is not at its lowest extent, motor 1802 lowers the platform. Using this logic, container 320 (if within the operating range of holder 1800) will be moved until the top of container 320 is between the elevation of sensors 2340 and 2341. Optionally, retainer 1805 can be used by the detection system to prevent container 320 from rising off of platform 1801 during operation. This may be necessary, for example, if container 320 is sealed; probe 150 has gone through the seal to aspirate sample; and probe 150 moves back out of the seal. As probe 150 moves out of the seal, friction between the probe and the seal may try to lift sample container 320. This lifting can be prevented by retainer 1805. Motion of retainer 1805 can be fully automated, fully manual, or partially automated; for example, retainer 1805 may automatically close when a container reaches the desired elevation between sensors 2340 and 2341 while being manually opened by the operator upon completion of the measurements.

2. Multiple Sample Holder

In some embodiments, the detection system may accommodate multiple holders 321 simultaneously. By accommodating multiple sample containers simultaneously, the operator may enjoy greater walk-away time. In some embodiments, the detection system can enable the operator to replace sample containers after the sample has been aspirated but before the measurement has completed. In this case, detection system through-put (i.e., measurements per hour) may not be substantially reduced from embodiments accommodating multiple sample containers simultaneously, while lowering detection system complexity.

In some embodiments, holder 321 may be a rotary disk accommodating 4, 5, 6, 7, 8, 9, 10, or more sample containers (e.g., container 1321) simultaneously.

In some embodiments, at least one multi-well reagent container can comprise an empty, non-assay well that is the sample container. In some embodiments, the operator may pipette the sample directly into this well.

K. Sample Pre-processing

In some embodiments, the sample matrix and/or environmental matrix may interfere with the measurement of the analyte of interest. For example, the sample matrix may bind to the analyte of interest in such a way as to compete with the binding reagents. In some embodiments, the desired units of the measurement can be the amount of analyte per volume of a subvolume of the sample, rather than the amount of analyte per volume of the sample. For example, the desired units for many blood-based tests is the amount of analyte per volume of plasma rather than per volume of whole blood.

1. Centrifugation

In some embodiments, components of the sample matrix and/or environmental matrix can be removed by centrifugation. In some embodiments, the detection system can comprise a centrifuge that can centrifuge the sample in the sample container to separate the sample by density. In some embodiments, the less-dense portion of the sample (e.g., plasma in the case of whole blood) can be used in the binding reactions. The centrifuge can be, for example, a StatSpin® MP (or other products also by Iris Sample Processing, (Westwood, Mass.)) that is integrated into the detection system. Other centrifuges can be based on U.S. Pat. No. 6,398,705 and/or U.S. patent application Publication No. 2004/0147386.

2. Filtration

In some embodiments, sample pre-processing includes filtration. Components of the sample matrix and/or environmental matrix can be removed by filtration. For example, blood samples can be applied to a filter membrane and a plasma sample can be generated in one region of the membrane. Similar matrix removals can be similarly accomplished with filters.

The pore size rating of the filter can vary depending on the matrix to be excluded. For example, 0.1 μm filters may be used to exclude viruses and larger particles. A 1 μm filter may be used to exclude spores and larger particles. A 3 μm filter may be used to exclude red blood cells and larger particles. A 5 μm filter may be used to exclude dirt particles and larger particles. A filter can block at least 90% of particles whose characteristic dimension is greater than its pore size rating. In some embodiments, the invention can use a filter device with a pore size rating of 0.05, 0.1, 0.2, 0.5, 1, 2, 3, 4, 7, 10, 15, 20, 50, or 100 μm to remove interfering components of the sample matrix. In some embodiments, the invention can use a filter with a pore size rating (a) greater than or equal to 0.1 μm and less than or equal to 4 μm; (b) greater than or equal to 0.02 μm and less than or equal to 0.1 μm; greater than or equal to 4 μm and less than or equal to 100 μm; and/or greater than or equal to 1 μm and less than or equal to 3 μm.

In whole blood samples, a fibrous web filter can be used as a size exclusion matrix. Plasma can move through this matrix without restrictions; however, particles above a certain size can have impeded flow. The fiber size and spacing between fibers can be designed to impede particles, such as the cellular components in blood. The movement of red blood cells (RBC) can be slowed down, but not trapped or immobilized. This may help prevent shear-induced lysis of the RBCs. White blood cells (WBC) are known to be very sticky and adhere to the fibrous media. Platelets may not be significantly impeded. Smaller objects like bacteria, viruses, proteins, or protein complexes can move freely through the fibrous matrix.

An asymmetric pore membrane filter can be used, for example, to remove cellular components from whole blood samples and generate plasma for analysis. This type of membrane filter has the pores change size across the membrane; from larger than blood cells to smaller than blood cells. In a specific embodiment, one side of the membrane would have pores 10 microns in size, while the other side would have pores 1 micron in size; as a whole, the membrane would have an overal pore size rating of 1 μm. Since the pore size changes gradually, the cellular components are not subjected to large shear forces and become trapped in a transition layer without lysing. The membrane region with smaller pores become enriched with plasma and depleted of cellular components.

The asymmetric pore membrane has advantages over fibrous web filters in the amount of area needed to separate plasma, particularly if the volume of plasma needed is small. The asymmetric pore membrane filter can be considered a dead end filter in which cellular components are trapped within the membrane and plasma can flow out of the membrane. Thus, this type of membrane can be highly efficient until the amount of trapped cells clogs the pores and slows flow to very slow rates. Therefore, plasma yields are a function of membrane projected surface area and level of clogged pores.

Conversely, the fibrous web filters use a wicking based size exclusion chromatography to effect plasma separation, in which the cellular components will eventually wick out of the filter. The amount of plasma generated will be a function distance wicked through this type of separation media.

There are many different methods in the filtration art to make filters. For example, metal wire is commonly used to make woven screens that can be used to catch extremely large particles, such as particles over 50 microns in size. To capture smaller particles, smaller diameter metal wire screens can be used, but they have limitations due to impedance to air flow (pressure drop).

Polymer-based membranes may be used to remove smaller particles from solutions. For example, nylon is often used in a phase inversion casting process to make membranes that range from a 10 micron pore size rating down to 0.1 micron pore size rating. Other polymer-based membranes (e.g. polyethersulphone, nitrocellulose, or cellulose acetate) are made by a solvent evaporation casting process.

Melt-blown polymer fibers can be used to make a fibrous web that acts as a filtration medium, in which fiber size, fiber spacing, and web thickness are tightly controlled. Other fibrous media, such as glass fiber, can be used as filtration membranes. The filtration medium is generally incorporated into a holding device that allows the fluid of interest to pass through the filter barrier in a controlled manner. In some embodiments, the invention can use a filter containing filtration media using the polymer polyethersulphone (PES). In certain embodiments, the PES filter can be encased in a plastic housing that can be (a) attached to a syringe, (b) part of a single use disposable designed to ease robotic automation, or (c) part of a multiple-use disposable designed to filter a plurality of samples. In some embodiments, melt-blown polymer fibers can be used to make a fibrous web that acts as a size-exclusion medium, particularly useful in the separation of blood cellular components from plasma.

Typically, particles smaller than the filter's pore size rating pass through a filter without hindrance, unless they are adsorbed to the filtration media. To prevent non-specific adsorption, filtration media can be surface-modified to reduce this type of interaction, e.g., by making the filter surface more wettable, i.e., more hydrophilic. It is generally believed that non-specific binding of analyte (that results in loss of recovery) is due to hydrophobic interactions, primarily through van der Waals type bonds. For example, coating the filtration media polyethersulphone (PES) with hydrophilic compounds like glycerol can increase the ability of water to wet the surface and can reduce analyte loss.

The coating agent can also be a protein. A suitable blocking protein may be bovine serum albumin (BSA), which can be dried onto the surface. Other blocking agents include nonionic detergents, such as Tween-20, Thesit (alkylpolyethyleneoxidePolyoxyethylene 9 lauryl ether), or alkyl-glucopyranoside.

Other methods to reduce non-specific absorption include, but are not limited to, free-radical polymerization, ion-beam initiated polymerization, ionizing radiation induced polymerization, plasma etching, and chemical coupling. These processes incorporate molecules with a significant number of hydroxyl groups that promote water hydration and reduce hydrophobic interactions. The specific method of surface modification depends primarily on the chemical nature of the filtration material used in the filter device. For example, ionizing radiation can be used to induce grafting of hydroxy-propyl-acrylate moieties onto nylon filtration media to render it hydrophilic and low protein binding. In some embodiments, the invention can use filtration media comprising the polymer polyethersulphone.

In some embodiments, filters can have chemical moieties attached to the surface to specifically bind interfering components. The filtration media can be covalently coupled to molecules having high affinity interactions with classes of molecules that are known to interfere with the immunoreaction or the detection methodologies. For example, molecules like lectins, which will bind to surface groups on red blood cells, or ethylenediaminetetraacetic acid (EDTA), which bind metal ions that could interfere with the detection process, can be attached to the filtration media.

Analysis of the plasma sample generated by filtration-based separation has usually been done within the separation membrane, or wicked into in an adjacent matrix. Consistent with the principles of the present invention, however, the filtrate can be removed from membranes so that it can be aspirated or dispensed by pipettor 405. To aid in the separation process and to recover free flowing plasma, an external pressure gradient can be applied to a blood sample within the filter media. The external pressure gradient increases flow rates, and if controlled within known parameters, can be used to recover plasma out of the filter media without contamination by blood cellular components or the lysed contents of these cells. The controlled use of pressure has defined ranges of action. When no pressure gradient is applied, only wicking type flow can occur, which can be limited by viscous drag forces or wetting rates. As the pressure gradient is increased, flow rates typically increase, but liquid may not flow out of a filter membrane. To induce liquid flow out of a membrane, the pressure gradient can be maintained above a minimal value, which can be called the flow pressure point. As the pressure gradient is increased above the flow pressure point, liquid can flow out of a membrane if liquid is available to flow in.

The values for the flow pressure point can vary according to the membrane construction, and can range from 0.5 psi to 1.5 psi. Below a pressure level called the bubble point, flow can stop when all the liquid available to flow in has entered the membrane filter. Above the bubble point, air can enter the wetted membrane and displace the contents. The values for the membrane bubble point can vary depending on membrane construction, fluid surface tension, fluid viscosity, and can range from 5 psi to 10 psi for blood separation membranes. High pressure gradients can impart high shear forces on the blood sample and cause lysis of the red blood cells. Therefore, preferred pressure gradients for blood samples can range from 0.5 psi to 5 psi, depending on time constraints, plasma yield volumes, and red blood cell lysis. Non-blood samples may withstand a larger range of available pressures, from for example, 0.04 psi to 15 psi or in some embodiments, 50 psi or less.

According to the embodiments disclosed herein, multiple mechanisms can be used to create a pressure difference across the filter. Positive gauge pressure can be applied upstream of the filter by gravity or by pump 870 or negative gauge pressure (vacuum) can be applied downstream of the filter by pump 870. Combinations of both negative and positive pressure can be used to produce a pressure gradient across the filtration media.

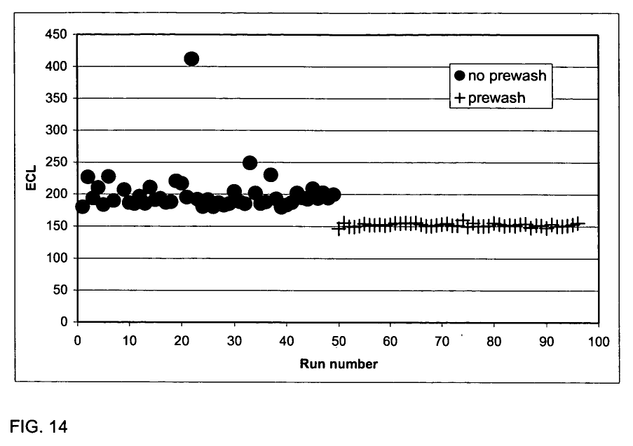

In some embodiments, the detection system can filter the sample so that only the filtrate is used in the measurement process. For example, as shown in FIG. 9, the system can use a filter 161 inside a disposable probe tip 160 on probe 150, aspirate sample through the filter, discard the filter, then dispense the filtrate into the at least one multi-well reagent containers. Pump 870 can be used to generate the pressure gradient across disposable filter 161. Optional pressure meter 162 can be used to achieve and/or monitor the pressure across the filter by monitoring the gauge pressure in tubing 203. In some embodiments, disposable probe tip 160 can be detachably connected to a multi-well reagent container for ease of inserting fresh tips into the detection system. In some embodiments, disposable probe tip 160 can be attachable to a multi-well reagent container for ease of tip disposal. In some embodiments, disposable probe tip 160 can be detachably connected and reattachable (after use) to a multi-well reagent container.

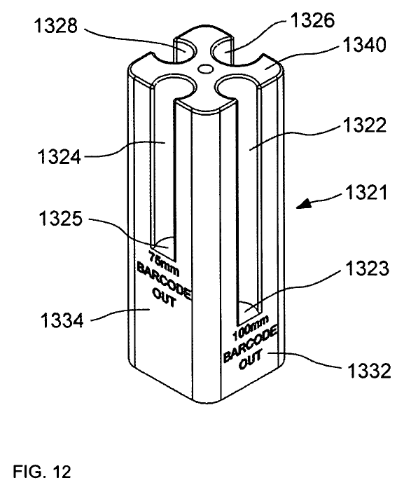

In some embodiments, holder 115 may accommodate a filter cartridge comprising at least one filter well 1150. The filter cartridge may be part of the multi-well reagent container. FIG. 7 displays some embodiments of filter well 1150. In some embodiments, sample can first enter air space 157 via opening 155, can be filtered by filter 152, and the filtrate can enter air space 156. Pipettor 405 can then collect the filtrate via opening 156. The bottom of the filter well can be arranged in such as way to enable pipettor 405 to aspirate almost all of the filtrate. In some embodiments, air space 157 and filter 152 can be annular rings, with opening 156 located in the center of the annulus. In some embodiments, sample can first enter air space 151 and filtrate can enter air space 153 and then flow into collection area 158 via connector 159. Pipettor 405 can then contact the filtrate without contacting filter 152.

To accelerate the filtration process, the air space 151 or 155 of the filter well 1150 that receives the sample may be sealed and pressurized, and the air space 153 or 156 that receives the filtrate can be vented. In some embodiments, to accelerate the separation process, the air space 158 or 156 that receives the filtrate can be sealed and a negative pressure applied while air space 151 or 155 can be vented.

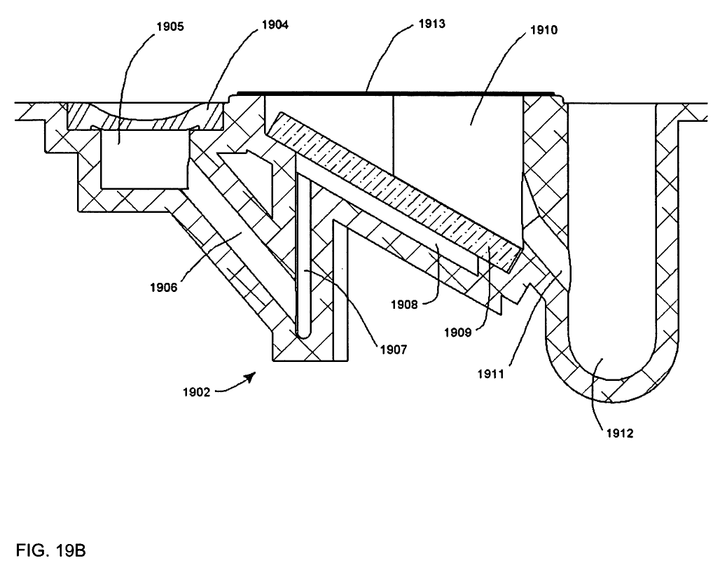

FIG. 19A and 19B depicts an exemplary embodiment of a multi-well reagent container comprising a filter cartridge 1902. Container 1901 holds 4 vessels (the seals 1705 are visible) in receptacle 1903. In operation, probe 150 would puncture seal 1904 and dispense the sample to be filtered (e.g., whole blood) into air space 1905. The sample to be filtered would travel through air space 1906, air space 1907 and air space 1908 to get to the filter 1909. By applying positive pressure, filtrate can be formed through by filter 1909 in air space 1910. Filtrate would flow via gravity down channel 1911 into collection area 1912. Probe 150 can then aspirate filtrate from collection area 1912. Seal 1904, by forming a seal around probe 150, enables pump 870 to create a pressure differential across filter 1909. Filter 1909 can be sealed into place, for example, by ultrasonic welding. Optional cover 1913 protects filter 1909 and reduces exposure of the sample to the exterior of container 1901. In another embodiment, seal 1904 instead covers collection area 1912. Unfiltered sample can be dispensed into air space 1905. Afterwards, probe 150 can puncture seal 1904 and pump 870 can create a negative gauge pressure across filter 1909 to generate filtrate.

3. Extraction Buffers

In some embodiments, sample pre-processing can include the use of extraction buffers. These buffers, for example, can be stored in liquid form in the multi-well reagent container. The recovery and detection of an analyte from a sample containing an interfering matrix can be increased by the use of appropriate extractions buffers. In some embodiments, the invention can use extraction buffers containing, for example, sodium borate, sodium chloride, and nonionic detergents to increase analyte recovery. Alternative buffers include sodium acetate, sodium malate, sodium oxalate, sodium citrate, sodium sulfate, sodium phosphate, as well as the potassium and lithium salts of borate, chloride, acetate, malate, oxalate, citrate, sulfate, and phosphate.

Extraction buffers can have various ionic strengths and pHs. A portion of the analyte of interest can be associated with the sample matrix through low affinity, non-specific interactions. These types of interactions can include, e.g., both ionic and hydrophobic bonding. In some embodiments, the ionic interactions between an analyte and matrix particles can be reduced by increasing the overall ionic strength of the extraction buffer(s), so that the mobile solution ions pair with the ionic surface charges of matrix particles, thereby promoting displacement of the analyte from matrix particles. In various embodiments, the pH of the solution can be changed from neutral (i.e., about pH 7) to either high pH or low pH to augment the ionic strength, to help reduce non-specific ionic interactions. Since most environmental matrix particles have a preponderance of negative surface charges, certain embodiments of the invention can use a high pH to ionize surface groups so that the extraction buffer can displace the analyte from the matrix particles. In some embodiments, the invention can use a buffer with pH of 8.5 and at least 0.5 molar sodium chloride. In some embodiments, the invention can use a buffer with a pH that is greater than or equal to 8.

In addition to ionic interactions, hydrophobic interactions can reduce analyte recovery. These types of interactions have been described as van der Waals types of interactions and can arise from the complex nature of water and hydrogen bonding. Ionic molecules can cause water molecules to form hydrogen-bonded cage structures (clathrates) around the charge groups, which tend to organize water molecules and reduce the movement of water molecules. Molecules with polar groups can dissolve in water by forming hydrogen bond structures between the hydrogen of water and the polar group. Portions of molecules that have neither ionic charges nor polar groups can be considered hydrophobic, and these portions tend to be driven together by exclusion from hydration events. Molecules with hydrophobic portions can be driven together to engage in van der Waals interactions. In this way, the overall structure of water can be stabilized.

To increase analyte recovery due to low-affinity, non-specific interaction with interfering matrix particles, certain embodiments of the invention can employ agents that cause a measured disruption of the water organization force. For example, hydrophobic interactions can be reduced by the use of detergents and chaotropic ions. Chaotropic ions are molecules that tend to disrupt the organizing force and structure of water. In some embodiments, nonionic detergents (e.g., Tween® 20) can be used to promote analyte recovery by binding the hydrophobic portion of the detergent molecule to the hydrophobic portions of the matrix and analyte. Various embodiments can use borate or other chaotropic ions to promote the disruption of the hydrogen-bonding structure of water. Borate ions are small and can constrain the water molecule cage structures more than most ions. Phosphate and sulfate ions can also be used in the invention. Some embodiments of the invention use one or more cations such as Mg²⁺, Ca²⁺, Li⁺, Na⁺, or K⁺. One skilled in the art will also appreciate that when using the divalent cations, additional unfavorable reactions may occur with some matrices. One skilled in the art will appreciate that, at a high concentration of chaotropic ions, the secondary and tertiary structures of protein molecules break down and high affinity interaction used in the immunoassay methods are disrupted. In some embodiments, the extraction buffer contains 0.1 M sodium borate (pH 8.5), 0.5 M sodium chloride, and 0.3% Tween® 20.

### III. EXAMPLES

### Example 1

### Filtered TSH Assay With and Without Prewash

The performance of the prewash mechanism was tested by constructing an assay for TSH (thyroid stimulating hormone), performing a free-bound separation using the prewash, and then measuring electrochemiluminescence (ECL) signal.

A. TSH Assay Construction

TSH standards were prepared by spiking human TSH into normal equine serum. The concentrations were calibrated by measuring the TSH levels using an Elecsys® 1010 (Roche Diagnostics Corporation, Indianopolis, Ind., USA).

Streptavidin-coated magnetizable beads (2.8 μm diameter), biotinylated anti-TSH capture antibody, and ruthenium tris-bipyridine anti-TSH labeled antibody reagents were obtained from Roche Diagnostics. All reagents, except the magnetizable beads, were filtered prior to use with a syringe filter (Gelman Laboratory) having a 0.2 μm pore size rating.

The assay was constructed using a 96 well microtiter plate. Into each well was pipetted 50 μL of TSH standard, 60 μL biotinylated antibodies, 50 μL labeled antibodies, and 40 μL of streptavidin-coated magnetizable beads.

The plate was incubated for 20 minutes at 37° C., followed by 30 minutes at room temperature. When the incubation was concluded, the plate was loaded onto a BioVeris M1M analyzer configured with a prewash mechanism.

B. Prewash

The prewash mechanism consisted of four sandwich magnets vertically positioned above the probe 150 and next to the tubing 203. In the normal or open state, the magnets were distant from the tubing. Beads drawn through the tubing would not be captured. In the closed state, the magnets would be fixed in direct contact with the tubing. Beads drawn through the tubing would be captured.

The prewash mechanism was implemented by closing the magnet state prior to the sample draw. As the sample was drawn into the tubing, the magnetizable beads were captured. Once the sample was drawn, the probe was raised, and the wash step initiated. From the instrument reservoir, wash buffer was dispensed through the tubing in the reverse direction as the sample draw. The wash buffer consisted of BioVeris BV-Glo-Plus solution. The beads were washed with 800 μL. The probe was then returned to the probe station.

In the case where the prewash is not used, the magnets remain in the open state at all times. The sample was drawn into the tubing in the same manner as when using the prewash. After the sample is drawn, the probe is raised and returned to the probe station.

C. ECL Readout

Once the plate was loaded onto the BioVeris M1M analyzer, each well was read consecutively by drawing 150 μL. The beads were first captured at the prewash, washed, and then released by returning the magnets to the open state. The beads were then drawn to an ECL detection module on the M1 Series. The beads were captured onto a working electrode and ECL was then initiated. The emission was detected by a photodiode (S1227-66BR; Hamamatsu Corporation, Bridgewater, N.J., USA).

D. Comparison of Prewash to Routine Assay

As a means to assess the performance of the prewash step, the TSH assay was run in triplicate with the prewash and compared to a TSH assay run without using the prewash. Table 1 below shows the comparison of the mean signals.

TABLE 1

| TSH concentration, | ECL signal | ECL signal |
|---|---|---|
| mlU/L | using prewash | without prewash |
| 0 | 166 | 198 |
| 0.14 | 315 | 312 |
| 0.45 | 683 | 600 |
| 1.75 | 2299 | 1771 |
| 8.7 | 11237 | 8211 |
| 44.0 | 53876 | 39211 |
| 89.5 | 104900 | 75535 |

The prewash TSH assay results showed a significant improvement over the routine TSH assay. At the zero TSH concentration, the ECL signal from the prewashed TSH assay was lower then the routine assay. The assay-specific signal with the prewashed TSH assay was greater then the routine TSH assay.

### Example 2

### Unfiltered TSH Assay with and Without Prewash

The performance of the prewash mechanism was tested by running samples that had high levels of interfering aggregates.

A TSH assay was constructed as shown in Example 1. In this example, only the zero level of TSH antigen was used, and the reagents were not filtered. The zero level sample represents an assay background. It was desired that the assay background signal level have a low mean and be very precise (as measured by the relative standard deviation, % CV).

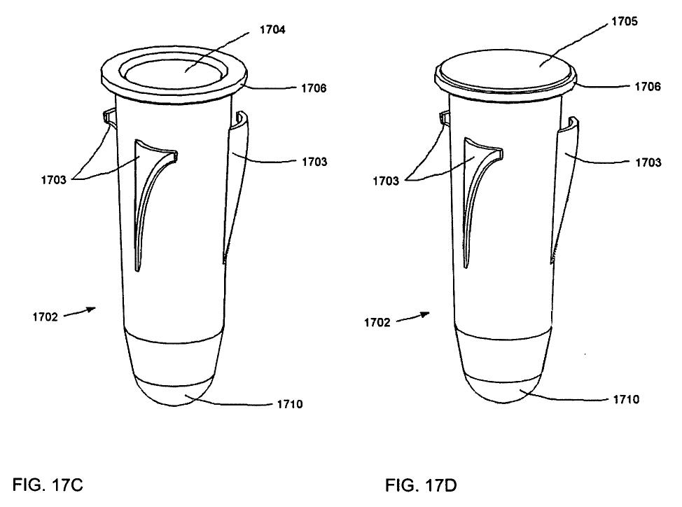

The routine TSH assay was run in replicates of 48. The mean ECL value was 201 with a % CV of 17.1%. The prewash TSH assay was run in replicates of 47. The mean ECL value was 153 with a % CV of 1.6%. These data are plotted in FIG. 14.

By implementing a prewash mechanism, the interfering aggregates were removed, as shown by the lower mean and lower % CV.

### Example 3

### Troponin T (TNT) Assay

A troponin T assay was performed using various aspects of the invention. Hardware similar to that of FIG. 1 was created. The prewash apperatus 220 of FIG. 4 and FIG. 5 was used. A multi-well reagent container was fabricated and used 750 μL vessels for all assay wells. The multi-well reagent container had the capacity for 40 assay wells. The multi-well reagent container also served as the sample container holder 321, which had capacity for 40 sample containers. The temperature of the assay wells was controlled using a 24.6 Ω power resistor (part HK5405R24.6L12B, Minco; Minneapolis, Minn., USA) and a Heaterstat™ controller (Minco; Minneapolis, Minn., USA) connected to a 15 V supply. Temperature control was isolated to the 40 assay wells and the 40 sample containers were left at ambient conditions. Other aspects of the instrumentation were taken from an M1M instrument (BioVeris Corporation, Gaithersburg, Md.), and custom software was developed to control the instrument.

For the assay wells, biotinylated monoclonal antibodies directed to TNT, Ru(bpy)₃²⁺labeled monoclonal antibodies directed to TNT, and 2.8 mm diameter streptavidin coated magnetizable beads (all from Roche Diagnostics, Switzerland) were mixed together for 1 hour on a rotator. To each 750 μL vessel, 200 μL of this solution was added and then placed on dry ice to quick freeze the solution. The vessels were freeze-dried overnight, and then stored at 4° C. in a low humidity environment until used. When needed, the vessels were placed in the receptacle of the multi-well reagent container.

For the samples, six levels of calibrators were created with the following concentrations of TNT: 0 ng/mL (Cal A), 0.65 ng/mL (Cal B), 6.13 ng/mL (Cal C), 12.2 ng/mL (Cal D), 24.5 ng/mL (Cal E), 43.4 ng/mL (Cal F). Controls were purchased from Bio-Rad Labs, and serum was spiked with different amounts of recombinant TNT for additional controls. The Hook samples were serum spiked with very large amounts of recombinant TNT, to ensure that signal levels show a hook effect.

The detection system pipetted 100 μL of each sample into an assay well. The assay wells were controlled to a temperature of 40.6° C. Carrier 302 agitated the multi-well reagent containers for 5 minutes at 20 Hz, using velocity profile 852 and an amplitude of 3 mm peak-to-peak. After the incubation, 100 μL was aspirated by pump 870 into probe 150. The magnetizable beads were captured in the prewash apparatus 220, and the sample matrix was dispensed back into the vessel. The beads were then transferred by pump 870 into flow cell 192, where they were captured. Electric potential was applied to electrodes in flow cell 192 to initiate electrochemiluminescence, and the luminescence was measured by a photodiode. Each sample was incubated for five minutes before aspiration out of the assay well. Samples were run in duplicate. A 4-PL curve was fit to the 6 calibrator data, and control samples were backfit and compared to the acceptable ranges to access quantitation.

As shown in Table 2, the coefficient of variation (CV) is given as well as the raw signal levels (ECL counts) and the predicted quantitation. Because there are 6 measurements for the 4 degrees of freedom in the curve fit, it is possible to have poor quantitation of the calibrators. These data, however, show excellent quantitation of the calibrators.

TABLE 2

| | | | | | % |
|---|---|---|---|---|---|
| Calibrators | | | Quantitation | % | quantitation/ |
| (ng/mL) | ECL counts | % CV | (ng/mL) | CV | target |
| Cal A (0) | 149 | 2.9 | Not | N/A | N/A |
| | | | detectable | | |
| Cal B (0.65) | 1671 | 6.0 | .65 | 5.5 | 100 |
| Cal C (6.13) | 21907 | 5.0 | 6.07 | 4.2 | 99 |
| Cal D (12.2) | 49198 | 0.58 | 12.1 | 0.5 | 99 |
| Cal E (24.5) | 114047 | 7.8 | 25.2 | 6.9 | 103 |
| Cal F (43.4) | 205564 | 1.7 | 42.9 | 1.5 | 99 |

The control results are shown in Table 3. One of the BioRad controls quantitated with the target range, while the other two backfit just outside the target range. The serum controls all quantitated within the target range. The hook samples all generated ECL counts well above Cal F, and so were appropriately reported as “out of range” (OOR), and thus no high dose hook effects were seen.

TABLE 3

| | ECL | | Quantitation | % | target | range |
|---|---|---|---|---|---|---|
| Controls | counts | % CV | (ng/mL) | CV | (ng/mL) | (ng/mL) |
| BioRad 1 | 535 | 0.55 | 0.21 | 0.64 | 0.32 | 0.16-0.48 |
| BioRad 2 | 2612 | 0.11 | 0.98 | 0.10 | 1.87 | 1.31-2.43 |
| BioRad 3 | 9645 | 2.3 | 3.02 | 2.0 | 5.30 | 3.70-6.90 |
| Serum 1 | 2273 | 0.63 | 0.86 | 0.56 | 0.79 | 0.63-0.95 |
| Serum 2 | 36081 | 3.5 | 9.28 | 3.0 | 8.26 | 6.61-9.91 |
| Serum 3 | 75038 | 6.6 | 17.4 | 5.7 | 14.8 | 11.8-17.8 |
| Hook 1 | 554271 | 2.3 | OOR | | 125 | 100-150 |
| Hook 2 | 779089 | 1.8 | OOR | | 258 | 206-310 |
| Hook 3 | 821575 | 3.8 | OOR | | 456 | 365-547 |

The precision of both the calibrators and the control samples were all below 7% in quantitated concentration.

All references cited herein are incorporated by reference in their entirety. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

It will be apparent to those skilled in the art that various modifications and variations can be made in the disclosed detection device, components, and methods without departing from the scope of the invention. Other embodiments of the invention will be apparent to those skilled in the art from consideration of the specification and practice of the invention disclosed herein. It is intended that the specification and examples be considered as exemplary only, with a true scope of the invention being indicated by the following claims and their equivalents.

1. A biological detection system used to measure one or more analytes of interest possibly present in a sample through the use of binding reactions comprising:
a detector configured to detect a label used in the binding reactions;
a holder configured to hold at least one multi-well reagent container;
at least one multi-well reagent container comprising the label and binding reagents for a plurality of binding reactions;
a probe configured to distribute a portion of a sample into at least one of the multi-well reagent containers;
one pump fluidically connected to the probe; and
a liquid-level detector for determining the presence and/or amount of liquid in at least one of a sample container and the multi-well reagent container.

2. The system of claim 1, further comprising an electrical energy source and an electrode, both suitable for initiating electrochemiluminescence.

3. The system of claim 1, further comprising a flow cell, wherein the flow cell comprises the detector and the flow cell fluidically connected to the probe and pump.

4-5. (canceled)

6. The system of claim 1, further comprising a filter configured to filter the sample to form a filtrate, wherein the filtrate is used in the binding reactions.

7-32. (canceled)

33. The system of claim 1, wherein the holder for the at least one multi-well reagent container has a capacity to hold at least 2 of the containers.

34. The system of claim 33, wherein the holder for the at least one multi-well reagent container has a capacity to hold at least 6 of the containers.

35-37. (canceled)

38. The system of claim 1, wherein the multi-well reagent container has identical reagents in at least 2 wells.

39-52. (canceled)

53. The system of claim 1, wherein the multi-well reagent container comprises a well that holds binding reagents specific for at least 2 of the analytes of interest.

54-55. (canceled)

56. The system of claim 53, wherein the multi-well reagent container comprises a well that holds binding reagents specific for at least 4 of the analytes of interest.

57-62. (canceled)

63. The system of claim 1, wherein the binding reagents comprise magnetizable beads; and wherein the system further comprises at least 2 magnetic capture zones.

64. The system of claim 63, wherein at least one of the magnetic capture zones is located fluidically between the tip of the probe and the flow cell.

65-84. (canceled)

85. The system of claim 2, further comprising:
a temperature control system used to regulate the temperature of the at least one multi-well reagent container and/or a location of the label when detected by the detector;
an ECL coreactant selected from piperazine-1,4-bis(2-ethanesulfonic acid); tri-n-propylamine; N,N,N′,N′-Tetrapropyl-1,3-diaminopropane; or salts thereof; and
an agitator configured to agitate the at least one multi-well reagent container;
wherein the label comprises a ruthenium chelate, an osmium chelate, or a mixture of both, wherein the system comprises one probe, the probe being part of the liquid level detector; wherein the binding reagents are dry and comprise magnetizable beads having a diameter ranging from about 0.4 microns to about 3 microns, and wherein the detector is a light detector.

86-91. (canceled)

92. A method to measure an analyte of interest possibly present in liquid in a sample container comprising:
(a) forming a composition in a well comprising a sample of optionally processed liquid from the sample container and binding reagents comprising a plurality of magnetizable beads, a plurality of labels, and a plurality of reagents specific for the one or more analytes of interest;
(b) incubating the composition to form complexes among the label, analyte of interest, and the plurality of magnetizable beads;
(c) separating any non-complexed label composition and sample matrix from the complexed label using a method comprising:
(i) aspirating the incubated composition from the well;
(ii) capturing the magnetizable beads with a magnet; and
(iii) dispensing the non-captured label composition into a waste location;
(d) releasing the captured magnetizable beads;
(e) transporting the magnetizable beads to a measurement zone; and
(f) detecting the complexed label to measure the concentration of the analyte of interest.

93-97. (canceled)

98. The method of claim 92, wherein the binding reagents are dry.

99. The method of claim 98, wherein the dry binding reagents are rehydrated solely by the sample.

100. The method of claim 92, further comprising
(iv) dispensing additional liquid into the waste location after step (iii) and before step (d).

101. The method of claim 92, wherein the waste location is the well.

102-105. (canceled)

106. The system of claim 3, wherein the flow cell is configured to measure radioactivity, optical absorbance, magnetic materials, magnetizable materials, light scattering, optical interference, surface plasmon resonance, luminescence, or a combination of any of the foregoing.

107-119. (canceled)

120. A biological detection system used to measure one or more analytes of interest possibly present in a sample through the use of binding reactions comprising:
a detector configured to detect a label used in the binding reactions;
a holder configured to hold at least one multi-well reagent container;
a probe, the probe configured to at least distribute a known amount of sample into at least one of the at least one multi-well reagent containers;
a pump, fluidically connected to the probe; and
2 or more magnetic capture zones fluidically connected and configured to collect and release magnetizable beads.

121. The system of claim 120, wherein at least one of the 2 or more magnetic capture zones is located fluidically between the tip of the probe and a location wherein the label is detected by the detector.

122. The system of claim 120, wherein the magnetic capture zones are configured to collect and release magnetizable beads that have a diameter ranging from about 0.4 microns to about 3 microns.

123. The system of claim 120, further comprising an electrical energy source and an electrode, both suitable for initiating electrochemiluminescence.

124-125. (canceled)

126. The system of claim 120, further comprising a filter configured to filter the sample to form a filtrate, wherein the filtrate is used in the binding reactions.

127-148. (canceled)

149. The system of claim 123, further comprising:
a temperature control system used to regulate the temperature near the at least one multi-well reagent container and/or a location of the label when detected by the detector;
an ECL coreactant selected from piperazine-1,4-bis(2-ethanesulfonic acid); tri-n-propylamine; N,N,N′,N′-Tetrapropyl-1,3-diaminopropane; or salts thereof; and
an agitator configured to agitate the at least one multi-well reagent container;
wherein the label comprises a ruthenium chelate, an osmium chelate, or a mixture of both, wherein the system comprises one probe, the probe being part of the liquid level detector; the magnetic capture zones are configured to collect and release magnetizable beads that have a diameter ranging from about 0.4 microns to about 3 microns, and wherein the detector is a light detector.

150-152. (canceled)

153. A multi-well reagent container comprising:
one or more vessels comprising binding reagents specific for an analyte of interest, a vessel opening through which the binding reagents enter and leave the vessel, and a vessel bottom which is the most distant part of the vessel from the vessel opening; and
a receptacle adapted to receive each of said one or more vessels comprising zero or more reagent cavities;
wherein one or more vessels are physically separate parts that are installed into the receptacle and are held in the receptacle via an attachment retention member; wherein the number of wells is considered to be the sum of the number of reagent cavities and number of vessels; and wherein the number of wells is 2 or more.

154-155. (canceled)

156. The multi-well reagent container of claim 153, wherein the attachment retention member is a snap fit.

157. (canceled)

158. The multi-well reagent container of claim 153, wherein the attachment retention member is configured so that the vessel bottom can move a greater distance than the vessel opening.

159. The multi-well reagent container of claim 153, wherein the vessel bottom forms part of the exterior of the multi-well reagent container.

160. The multi-well reagent container of claim 153, wherein the vessel opening is covered by a seal that is pierceable by a probe.

161-162. (canceled)

163. The multi-well reagent container of claim 153, wherein the number of vessels ranges from 2 to 36.

164-172. (canceled)
